(19) United States
(12) Patent Application Publication
(10) Pub. No.: US 2014/0358447 A1
(43) Pub. Date: Dec. 4, 2014

(54) INSTANT, IN-SITU, NONDESTRUCTIVE MATERIAL DIFFERENTIATION APPARATUS AND METHOD

(71) Applicants: Timothy Edwin Doyle, Orem, UT (US); Laurel Ann Thompson, Alpine, UT (US); Leila Jannesari Ladani, Willington, CT (US)

(72) Inventors: Timothy Edwin Doyle, Orem, UT (US); Laurel Ann Thompson, Alpine, UT (US); Leila Jannesari Ladani, Willington, CT (US)

(21) Appl. No.: 14/264,233

(22) Filed: Apr. 29, 2014

Related U.S. Application Data
(60) Provisional application No. 61/963,993, filed on Dec. 21, 2013.
(60) Provisional application No. 61/964,885, filed on Jan. 17, 2014.
(60) Provisional application No. 61/964,884, filed on Jan. 17, 2014.
(60) Provisional application No. 61/964,886, filed on Jan. 17, 2014.
(60) Provisional application No. 61/964,887, filed on Jan. 17, 2014.
(60) Provisional application No. 61/959,939, filed on Sep. 6, 2013.
(60) Provisional application No. 61/854,846, filed on May 3, 2013.

(51) Int. Cl. G01N 29/44; G01N 33/483

(52) U.S. Cl. CPC G01N 29/44; G01N 33/4833
USPC 702/19

(57) ABSTRACT

Specular, ultrasonic, piezoelectric, detection devices provide real-time, analytical, edge finding in tissues during tumor surgery. Piezoelectric probe sensors at high frequencies (e.g., 10 to 100 MHz) characterize microstructure of cells and tissues. Through-transmission or specular reflection enables nondestructive testing in real time. Peak density analysis in power spectra, second-order spectrum analysis measuring the slope of the Fourier transform of the power spectrum, artificial intelligence pattern recognition, and modeling interpret the results. Model-based data analysis may compare experimental data with a computer simulation. Such comparisons may be based upon pattern classifications, including principal component analysis (PCA). Combining the above detection devices and analytical methods provides speed, accuracy, simplicity, and nondestructive mechanisms that militate for reliable, real-time diagnosis of tumor margins, tissue pathology, cell phenotypes, and molecular subtypes.

### RELATED APPLICATIONS

This application claims the benefit of U.S. Provisional Patent Application Ser. No. 61/963,993 entitled ULTRASOUND MICROSENSOR FORCEPS FOR DETERMINING TISSUE PATHOLOGY, filed on Dec. 20, 2013; U.S. Provisional Patent Application Ser. No. 61/964,885 entitled HIGH-FREQUENCY ULTRASONIC SURFACE SENSOR FOR MICROSCOPIC ANALYSIS OF TISSUE PATHOLOGY, filed on Jan. 16, 2014; U.S. Provisional Patent Application Ser. No. 61/964,884 entitled DEVICE FOR ULTRASONIC AND OPTICAL PHENOTYPING OF BIOLOGICAL CELLS filed on Jan. 16, 2014; U.S. Provisional Patent Application Ser. No. 61/964,886 entitled ULTRASOUND MICRONEEDLE SENSOR FOR CHARACTERIZING BIOLOGICAL MATERIALS filed on Jan. 16, 2014; U.S. Provisional Patent Application Ser. No. 61/964,887 entitled ULTRASOUND MICROSENSOR SCALPEL FOR DETERMINING TISSUE PATHOLOGY, filed on Jan. 16, 2014; U.S. Provisional Patent Application Ser. No. 61/959,939 entitled OPTICAL METHOD FOR DETERMINING THE PATHOLOGY OF TISSUE AEROSOLS PRODUCED DURING SURGERY, filed on Sep. 3, 2013; U.S. Provisional Patent Application Ser. No. 61/854,846 entitled ULTRASONIC METHOD FOR DETERMINING THE MOLECULAR SUBTYPES OF CANCER, filed on May 1, 2013. All of the foregoing Provisional Patent Applications are hereby incorporated herein by reference.

### BACKGROUND

1. Field of the Invention

This invention relates to materials differentiation and, more particularly, to novel systems and methods for determining tissue margins, the edge or boundary between a cancerous cell colony and adjacent clean or healthy cells.

2. Background Art

Cancer influences nearly all persons and is often addressed by surgery, and may include additional treatments such as chemotherapy, radiation, and the like. It has been shown that the margin, meaning the margin between healthy tissues and cancerous tissues strongly correlates with long term survival. For example, if margins are tested positive (cancer cells are present in the margin of surgical incisions, or close), then the probability of disease-free survival is significantly reduced. A large portion of patients actually require re-excision because they test positive at the surgical margins.

In surgery for cancerous tumors, an ever present question is containment. Some tumors are quite well contained, well bounded, and are removable in their totality. Other tumors may have portions that extend into surrounding tissues with longer branches or fingers that are not well defined nor easily removed. In determining whether a tumor has been completely removed, a surgeon needs a mechanism to determine the constitution of the marginal or edge cells remaining after a surgical incision or procedure.

To this end, certain attempts have been made such as the work by Albert Migliori in a Resonant Ultrasound Spectroscopy; Patent Publication WO 1992005439 A1 published Mar. 29, 1992. However, such resonances arise from the macroscopic vibrations of the object and are dependent on that object's macroscopic properties such as the size, shape, and other physical properties of the particular object in question. Thus, organ boundaries, as between distinct, macroscopic objects may be detected by such mechanisms.

Likewise, certain of the inventors of the instant inventions have also done additional work as identified in United States Patent Publication U.S. 2013/0269441 published Oct. 17, 2013, which is incorporated herein by reference. Similarly, certain processes have been developed for sectioning and testing cellular samples or specimens at the time of surgery. However, such processes have such a long turnaround time that they lack effectiveness and utility during the time of surgery. They simply indicate whether additional surgery will be immediately forthcoming.

For example, in certain systems, electrical or laser cutters providing automatic cauterization of tissues produce smoke. The rapid vaporization of tissues puts off an aerosol of unburned, partially burned, or otherwise evaporated materials. It has been shown that gas chromatography from such an electrical scalpel may be evaluated by rapid evaporative ionization mass spectrometry (REIMS) to distinguish between malignant tissue and nonmalignant tissue.

Such testing from data gathered from the smoke produced by cauterizing surgery still requires more time than is actually available during surgery, and lacks the desired simultaneity with the surgery. The time of flight or passage within a conduit, sensitivity, aliquot size requirements, samplings, and light may be comparatively limited. Moreover, more conventional methods of analysis including liquid chromatography and the like also are far removed from the time of surgery and therefore only have limited utility.

Various methods have been introduced and investigated for pre-surgery and intra-surgery detection of margin sizes or of cancer in the surgical margins. Various methods relying on electromagnetics, such as CT and MRI (magnetic resonance imaging) have been used to test surgical margins in order to detect cancer threat. For example, terahertz imaging, Raman spectroscopy or scattering, optical coherence tomography, and diffuse reflectance spectroscopy have been used. Likewise, intraoperative pathology is also used for margin assessment. However, the analysis, whether by cytology, frozen section analysis, touch preparation cytology, or the like is still typically unable to identify close margins.

Since complete removal of cancerous cells is important to the prevention of recurrence, surgeons want a negative margin (cancer free). However, current techniques in conventional pathology may take days for accurate analysis of specimens. Moreover, the specimens are necessarily excised, and thus involve destructive testing.

Therefore, it would be an advance in the art to provide a faster, more accurate, real-time, reliable mechanism for testing a specimen, doing so nondestructively if possible, analyzing that specimen, and providing the analysis of positive or negative margin during actual surgery in order to provide immediate feedback in time to further expand or move the margin in single surgery, if possible, to remove all malignant tissue.

It would be a further advance in the art to provide algorithms, analytical methods, and processing techniques that will minimize the computational time required for analysis of data. Thus, it would be an advance to develop an improved method for testing specimens, for detecting differences in tissues, for analyzing both the characterizations and the evaluations at the time of surgery, and methods for modeling such malignancies.

### BRIEF SUMMARY OF THE INVENTION

In view of the foregoing, in accordance with the invention as embodied and broadly described herein, a method and apparatus are disclosed in one embodiment of the present invention as including a data acquisition method that employs both through-transmission measurements and specular reflection measurements. Either or both may be used. Typically, a comparatively short duration, on the order of two to four wave lengths, defines an ultrasonic pulse. The pulse is generated at a comparatively high frequency. That is, compared to prior art techniques, a much higher frequency is relied upon.

A pulse is generated and propagated through the material, by either through-transmission or specular reflection. The incident wave, having passed through the specimen material is then detected and recorded.

In certain embodiments, the detector may be a piezoelectric cell on the opposite side of the specimen from a piezoelectric transmission cell. In this situation, the detector cell records or detects for recording, the incident, through-transmission pulse. In another embodiment, a comparatively hard, rigid, stiff surface is positioned opposite the transmitter or transmission cell. Thus, a pulse from the transmission cell propagates through the specimen material, reflects off the hard anvil or reflector surface, and returns to be detected by the piezoelectric cell in agreement with the transmitter.

The specular reflection differs substantially from conventional diffused scattering technology. By relying on specular reflection, the actual microstructure of individual cells will provide a series of peaks, which peaks, by their numbers, density as described herein, at specific bands or within specific bands of frequency, and within specific bands of transformed domain (e.g., second order spectrum analysis) can be used to characterize the nature of tissues.

Actually more information of use in detecting margins can be determined while actually knowing comparatively less about the material. For example, different types of tissues may be characterized and documented according to their responses. Thus, exactly why the response is what it is may become less important than the fact that a response is identified with a particular type of tissue. Thus, the question of tissue type (e.g., healthy or malignant) may be answered, directly by the characterization, rather than spending more analysis on exactly why the characterization differs. However more detail may be used in modeling, as discussed hereinbelow.

In one presently contemplated embodiment, a through-transmission measurement uses two ultrasonic transducers. One functions as a transmitter and one as a receiver. They are positioned facing one another on opposite sides of the specimen. By arranging the two transducers (transmitter and receiver) collinearly, they form a single axis, each plane being parallel and opposite to the other and perpendicular to the shared axis. Transmission of an ultrasonic beam from the transmitting transducer assures the best available reception of the transmitted beam at the receiving transducer.

The specimen is constituted by a non-invaded or other portion of tissue captured between the transducers. Typically, a fluid is positioned to maintain the maximum acoustic coupling between the transducers and the specimen. For example, the presence of any air gaps dramatically alters transmission of ultrasonic waves. Accordingly, a coupling fluid is recommended. Similarly, it may be valuable or necessary to isolate the specimen material from the actual transducers by providing a sanitary, migration, or electrical barrier. This may be constituted by a barrier material against which the transducer is placed, or may be a coating over the transducer materials. This provides electrical, biological, chemical, and other isolation.

In certain embodiments, a specular reflection measurement may have a single transducer operating as both the transmitter and the receiver. Meanwhile, the opposite face of such a detector or the opposite platen or anvil is typically hard, flat, parallel, and rigid. Thus, it becomes a node for reflection of the through-transmitted ultrasonic beam. Placement of the anvil perpendicular to the transmitted ultrasonic beam optimizes reflection back to the transducer.

Again, the specimen material is placed between the transducer and the anvil resulting in a maximum reflection of the transmitted wave propagating through the specimen. Of course, isolation for chemical, dielectric, or other purposes may be accomplished by a thin film, a rigid material, a coating, or the like. Similarly, a coupling fluid may also be used between the transducer and any material in contact with the transducer in order to maximize transmissivity of the propagated waves at the surface of the transducer.

In certain embodiments, the ultrasonic wave is primarily specular in nature. In contrast to prior art systems that use diffuse reflections from anomalies or heterogeneities within a material, a specular reflection is intended to be integral. Thus, any deviation therefrom indicates alterations in the microstructure. Thus, materials that are homogenous are equally susceptible to characterization by an apparatus and method in accordance with the invention. In general, comparison is possible between any materials. Thus, dispersions, suspensions, slurries, colloids, composites, and materials with granular module structures, immiscible mixtures, and the like may all be suitably characterized by an apparatus, system, and method in accordance with the invention.

In certain embodiments, the invention may use both time-domain, waveform analysis and frequency-based, spectral analysis. For example, various measurement methods may be combined to analyze and differentiate materials. However, beyond such multivariate analysis, a system and method in accordance with the invention may obtain more than simple attenuation and wave speed information from time-domain data.

For example, principal component analysis, a comparison of spectra from various specimens, need not be the central point in preferred embodiments of systems in accordance with the invention. Rather, systems and methods in accordance with the invention may use several unique methods for analysis.

For one, determining the peak density of a frequency domain power spectrum identifies not just the existence of a peak at a frequency but rather a number of peaks within certain frequency bands. Applicants have learned that these peak densities resulting from specular waves are significant in characterizing the microstructure of tissues.

Similarly, determining the slope of a second Fourier transform of time domain data also has been found to characterize tissue samples uniquely according to their microstructure characteristics. The power spectrum may be thought of as the absolute value of the Fourier transform. Herein, this analysis technique will be referred to as second-order spectrum analysis.

Materials may also be compared to a computational model of themselves or of another material. Principal component analysis may be used on the experimental data in combination with simulated data. Such data is calculated from computational models of ultrasonic scattering. Modeling is at a microscopic level of cellular structures and material properties. This provides new insights and new characterizations of specimens.

For example, the first analysis of peak densities based on the physical phenomenon of materials with different microstructures will produce different spectral features (peaks and valleys) at varying frequencies due to the size, shape, and other ultrasonic properties of those microstructural features. Computational models have demonstrated that these spectral features arise from scattering and interference effects of the ultrasound at a microscopic level.

Moreover, testing has demonstrated to the inventors that a wide variety of biological specimens and even synthetic materials will demonstrate that the size, scale, level of complexity, and level of heterogeneity of the microstructures affect the number of peaks and valleys in the spectra. Thus, by evaluating the number of peaks or the “peak density” characterizing the number of peaks and valleys within a specified band of frequency of a power spectrum, any two different materials may be compared.

Actual frequencies of the peaks and valleys are not necessarily required to be determined. These will vary greatly in highly complex, heterogeneous materials. Likewise, it has been found that peak density analysis is particularly sensitive to highly heterogeneous biological tissues and synthetic materials. Thus, characterizations may be made without necessarily analyzing all of the actual frequencies of the peaks and valleys. That is, peak density analysis can be applied to biological materials and other materials. It can likewise be applied by through-transmission and specular reflection. Results are similar, although sensitivity and effectiveness appear to be better with through-transmission data.

In contrast to the work of investigators such as Migliori (Resonant Ultrasound Spectroscopy; Patent Publication WO 1992005439 A1 published Mar. 21, 1992) peak density analysis quantifies spectral features at higher frequencies to determine the geometric qualities of a materials microstructure. It does not require that the frequencies of each peak or valley be determined in order to characterize the microstructure of the material.

As to the second example, a second-order spectrum analysis relies on the slope or the derivative of a second Fourier transform. In this analytical method for differentiating subjects, a method may take a Fourier transform of a time-domain waveform. This may be an ultrasonic pulse of one of the types discussed hereinabove. Thereafter, a processor may take an absolute value of the transformed data in order to obtain a power spectrum. Next, the Fourier transform or “second transform” is now taken of the power spectrum. The absolute value of this second, transformed, data provides the second order spectrum.

By taking a maximum value found in the second order spectrum, and using it to divide into the data, one may normalize the data against that maximum value. Now, an independent variable is defined for this second order spectrum and is called quefrency. This number quefrency is the reciprocal of frequency. Frequency is a number of cycles per unit time and has units of inverse time. Accordingly, quefrency is the reciprocal of frequency and has units of time directly.

One may use numerous analytical techniques of curve fitting, line fitting, various numerical methods, and the like to differentiate or determine the slope of the second-order spectrum. That is, here the concept of differentiation is the mathematical concept from calculus of first order differentiation with respect to an independent variable. Thus, differentiation here means finding a slope, the rate of change, of the range with respect to the domain.

There exists an analysis referred to as cepstrum analysis that uses an inverse Fourier transform of the power spectrum. This is used to calculate a value referred to as the cepstrum. One will note that the reversal of the spelling of the first syllable identifies the cepstrum as this transform of the power spectrum. In contrast, second-order spectrum analysis uses the forward Fourier transform of the power spectrum to generate a second-order spectrum.

The second-order spectrum analysis is particularly sensitive to highly homogenous biological tissues and synthetic materials. Thus, the new second-order spectrum analysis in accordance with the invention may be applied to both through-transmission and spectrum reflection measurements with similar results. However, in contrast to peak density analysis, specular reflection data is somewhat more effective.

In certain embodiments of an apparatus and method in accordance with the invention, model-based data analysis may compare experimental data with model data calculated from a computer simulation. For example, in one embodiment of a computer program, a comparison may be based upon pattern classifications. This may even include principal component analysis (PCA). However, other comparisons of classification may also be used.

In a method in accordance with the invention, one approach may be to apply a microstructure-based computer simulation of cells and tissues to generate a model of ultrasonic data expected. That is, the material properties may be generated in a database.

For example, a range of cell or tissue phenotypes may include cell size, nucleus size, tissue microstructures, shear modulus, bulk modulus, and so forth corresponding to cellular components, and other material properties as distinct from the material properties of others. Thus, the material properties of different regions of a cell, including the dimensions of those regions, may all be modeled.

Mathematically analyzing the ultrasonic response of a propagated pulse or wave pulse through (or into and back from) such a structure may be very informative. The model may then be compared to experimental data. The experimental data may help to refine, calibrate, or otherwise more accurately characterize the material. Accordingly, a comparison of experimental data with the model may be used in order to classify experimental data.

Herein, the mention of tuning the model to data may actually be done before or after application of classification algorithms. For example, artificial intelligence engines may provide for comparison of experimental data to model data that has been collected and recorded, such as in a database. However, before or after such comparisons are made, experimental data may be compared. As a practical matter, one may conduct experiments to tune the model (e.g., adjust weighting of parameters or outcomes) to tweak or calibrate the model before doing a blind analysis of unknown tissues.

Data may be in the form of waveforms or of spectra. In certain embodiments, a method in accordance with the invention may rely on a multipole expansion technique effective due to its computational efficiency. However, other modeling techniques such as the finite element method (FEM), boundary element method (BEM), and finite-difference time-domain (FDTD) may also be used. Pattern recognition programs have been used successfully as the foregoing. For example, PCA may provide insights and analysis.

In other embodiments of apparatus and methods in accordance with the invention, various devices have been configured and designed to collect measurements as described hereinabove. For example, a micro-sensor forceps device, a double-needle, micro-sensor, and a surface-wave generator and detector has been considered as a probe. Likewise, a penetrating micro-needle, specular transmission device is another possible transmission and detection device. A microscopic scalpel, other surface sensors, and the like have been proposed hereinafter.

Likewise, a phenotyping cuvette may operate with suspensions of materials. In certain embodiments, a smoke-analysis, light-scattering device may also be used.

Typically, a scalpel, forceps, surface sensor probe, micro-needle probe, or the like will typically rely on two ultrasonic transducers to acquire through-transmission measurements. However, the micro-sensor scalpel, micro-sensor forceps, micro-needle sensors, and phenotyping cuvette may all introduce a hard, rigid surface or anvil from which specular ultrasonic measurements can be taken. Through-transmission of specular reflection measurements is particularly well suited to the data analysis and methods described hereinabove.

In many respects, the phenotyping cuvette may destructively and invasively characterize excised cells in a suspension. Thus, this device may be fundamentally different than other devices because it is dependent on destructive testing. The other devices need not destroy the specimen. In contrast, a cell suspension necessarily involves destruction of tissues to provide cells for evaluation. Also, such a system does not necessarily work on the principle of introducing a solid specular surface. It may be designed to use a comparatively low-frequency ultrasound to suspend the cells in a planar region over which a higher frequency ultrasonic pulse may be reflected. Thus, this specular surface of cells is created and used as the primary means of measuring cellular properties, same properties which are measured by the other devices.

Thus, an apparatus and method in accordance with the invention may comprise both a through-transmission as well as a specular reflection measurement system. Direct ultrasonic pulses may be propagated through a material, with their propagated wave pulse being analyzed at its destination (whether through-transmitted or reflected) in order to characterize the material. These measurements are unique and rely on specular waveforms rather than diffuse reflections, scattering, or the like.

A multi-variable parametric analysis obtains information about the evaluated material. Properties such as bulk modulus and attenuation, well known mechanical properties for materials may be used to characterize individual portions for modeling purposes. These also are well established herein to provide microstructural variations that will be detected and thus used to distinguish different tissues.

In the course of implementing the invention, certain unique methods for analyzing the structure of the power spectra (peak density analysis), the structure of the Fourier transform of the power spectra (second-order spectrum analysis), and the power spectra in comparison to model data (model-based, data analysis) are relied upon. Each may extract information directly related to the microstructure and properties distinct and unique to each material. The “high-frequency” ultrasound used in accordance with the invention has wave lengths on the same scale as cells and tissue microstructures and biological materials. The structures of the ultrasonic spectra and second-order spectra are sensitive to biological microstructures. In other words, the resolutions match and thereby define the useful frequencies and wave lengths. Meanwhile, the various novel devices for taking data improve the ability to do nondestructive testing instantaneously.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is a schematic block diagram of a computer system suitable for implementing methods and apparatus in accordance with the invention;

FIG. 2 is a schematic block diagram of one configuration of a computerized system in accordance with the invention;

FIG. 3 is a schematic block diagram of one embodiment of a process for testing in accordance with the invention;

FIG. 4 is a schematic block diagram of one embodiment of a method for peak density analysis in accordance with the invention;

FIG. 5 is a schematic block diagram of one embodiment of a process for second-order spectrum analysis in accordance with the invention;

FIG. 6 is a schematic block diagram of one embodiment of a model-based analysis method in accordance with the invention;

FIG. 7 is a side, elevation, cross-sectional view of one embodiment of forceps in accordance with the invention;

FIG. 8 is a side, elevation, cross-sectional view in schematic form of a surface probe operating in accordance with the invention;

FIG. 9 is a top view of one embodiment of a micro-needle probe in accordance with the invention;

FIG. 10 is a side, elevation, cross-sectional view thereof;

FIG. 11 is a side, elevation, cross-sectional view thereof in operation;

FIG. 12 is a top plan, cross-sectional view of one embodiment of a piezoelectric scalpel device in accordance with the invention;

FIG. 13 is a side elevation view thereof, having one prong of the scalpel removed for visibility of other details;

FIG. 14 is a top plan view of one embodiment of an ultrasonic digest chamber in accordance with the invention;

FIG. 15 is a side, elevation, cross-sectional view thereof;

FIG. 16 is a top plan view of an alternative embodiment thereof;

FIG. 17 is a side, elevation, cross-sectional view thereof;

FIG. 18 is a side, elevation, cross-sectional view of an alternative embodiment of an ultrasonic chamber for liquids, having a drop guide sleeve;

FIG. 19 is a side, elevation, cross-sectional view of an alternative embodiment thereof;

FIG. 20 is a side, elevation, cross-sectional view of an alternative embodiment thereof;

FIG. 21 is a schematic diagram of one embodiment of an optical method and apparatus for determining the pathology of tissue aerosols during surgery;

FIG. 22 is a chart illustrating an ultrasonic trace of a through-pulse signal through various samples, in a time domain;

FIG. 23 is a chart illustrating an ultrasonic trace of a pulse-echo signal thereof;

FIG. 24 is a chart illustrating a peak density trace of the data of FIG. 22, in the frequency domain;

FIG. 25 is a chart illustrating a peak density trace of the data of FIG. 23, in the frequency domain;

FIG. 26 is a bar chart illustrating the peak densities of data from assorted sizes of spheres in a matrix;

FIG. 27 is a bar chart illustrating the lack of correlation of relative attenuation coefficients to the assorted sizes of spheres;

FIG. 28 is a bar chart illustrating the lack of correlation of ultrasonic velocity to the assorted sizes of spheres;

FIG. 29 is a chart identifying the changes in ultrasonic response of porcine ventricle tissue upon continued, differential uptake of formalin by components of the micro-structure;

FIG. 30 is a schematic block diagram of a process for molecular sub-type and other classification;

FIG. 31 is a schematic diagram of a test set up for ultrasonically probing a monolayer culture of cells;

FIG. 32 is a diagram of a cell culture modeled for comparison;

FIG. 33 is a chart of the ultrasonic traces in a frequency domain showing the distinguishing of the cells with various changes in shear modulus;

FIG. 34 is a chart of the ultrasonic traces in a frequency domain showing the distinguishing of the cells with various changes in bulk modulus;

FIG. 35 is a chart of the ultrasonic trace in a time domain showing the reflection of an ultrasonic pulse in a matrix absent a cell culture;

FIG. 36 is a chart of the ultrasonic trace in a time domain showing the reflection of an ultrasonic pulse in a matrix containing a cell monolayer culture; and

FIG. 37 is a chart of the ultrasonic trace, in a frequency domain, of each of several different lines of cancer cells according to various features of their respective signal responses identifiable by shapes of the curves, which are identifiable by artificial intelligence engines and various mathematical constructs characterizing the curves.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

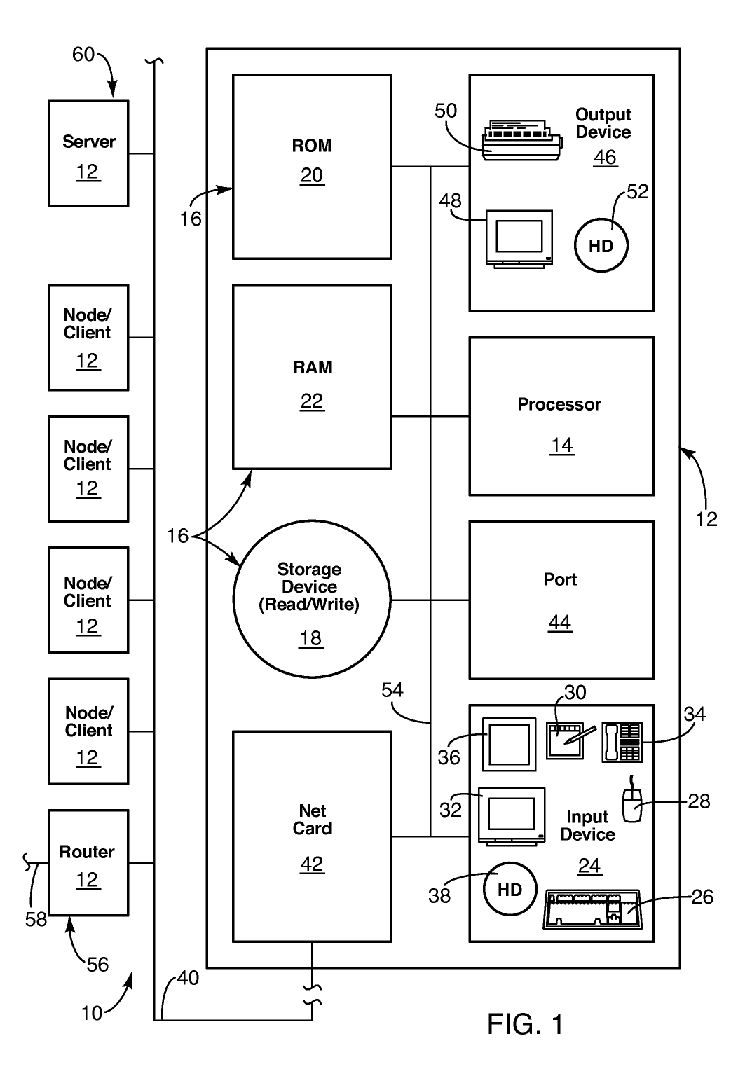

Referring to FIG. 1, an apparatus 10 or system 10 for implementing the present invention may include one or more nodes 12 (e.g., client 12, computer 12). Such nodes 12 may contain a processor 14 or CPU 14. The CPU 14 may be operably connected to a memory device 16. A memory device 16 may include one or more devices such as a hard drive 18 or other non-volatile storage device 18, a read-only memory 20 (ROM 20), and a random access (and usually volatile) memory 22 (RAM 22 or operational memory 22). Such components 14, 16, 18, 20, 22 may exist in a single node 12 or may exist in multiple nodes 12 remote from one another.

In selected embodiments, the apparatus 10 may include an input device 24 for receiving inputs from a user or from another device. Input devices 24 may include one or more physical embodiments. For example, a keyboard 26 may be used for interaction with the user, as may a mouse 28 or stylus pad 30. A touch screen 32, a telephone 34, or simply a telecommunications line 34, may be used for communication with other devices, with a user, or the like. Similarly, a scanner 36 may be used to receive graphical inputs, which may or may not be translated to other formats. A hard drive 38 or other memory device 38 may be used as an input device whether resident within the particular node 12 or some other node 12 connected by a network 40. In selected embodiments, a network card 42 (interface card) or port 44 may be provided within a node 12 to facilitate communication through such a network 40.

In certain embodiments, an output device 46 may be provided within a node 12, or accessible within the apparatus 10. Output devices 46 may include one or more physical hardware units. For example, in general, a port 44 may be used to accept inputs into and send outputs from the node 12. Nevertheless, a monitor 48 may provide outputs to a user for feedback during a process, or for assisting two-way communication between the processor 14 and a user. A printer 50, a hard drive 52, or other device may be used for outputting information as output devices 46.

Internally, a bus 54, or plurality of buses 54, may operably interconnect the processor 14, memory devices 16, input devices 24, output devices 46, network card 42, and port 44. The bus 54 may be thought of as a data carrier. As such, the bus 54 may be embodied in numerous configurations. Wire, fiber optic line, wireless electromagnetic communications by visible light, infrared, and radio frequencies may likewise be implemented as appropriate for the bus 54 and the network 40.

In general, a network 40 to which a node 12 connects may, in turn, be connected through a router 56 to another network 58. In general, nodes 12 may be on the same network 40, adjoining networks (i.e., network 40 and neighboring network 58), or may be separated by multiple routers 56 and multiple networks as individual nodes 12 on an internetwork. The individual nodes 12 may have various communication capabilities. In certain embodiments, a minimum of logical capability may be available in any node 12. For example, each node 12 may contain a processor 14 with more or less of the other components described hereinabove.

A network 40 may include one or more servers 60. Servers 60 may be used to manage, store, communicate, transfer, access, update, and the like, any practical number of files, databases, or the like for other nodes 12 on a network 40. Typically, a server 60 may be accessed by all nodes 12 on a network 40. Nevertheless, other special functions, including communications, applications, directory services, and the like, may be implemented by an individual server 60 or multiple servers 60.

In general, a node 12 may need to communicate over a network 40 with a server 60, a router 56, or other nodes 12. Similarly, a node 12 may need to communicate over another neighboring network 58 in an internetwork connection with some remote node 12. Likewise, individual components may need to communicate data with one another. A communication link may exist, in general, between any pair of devices.

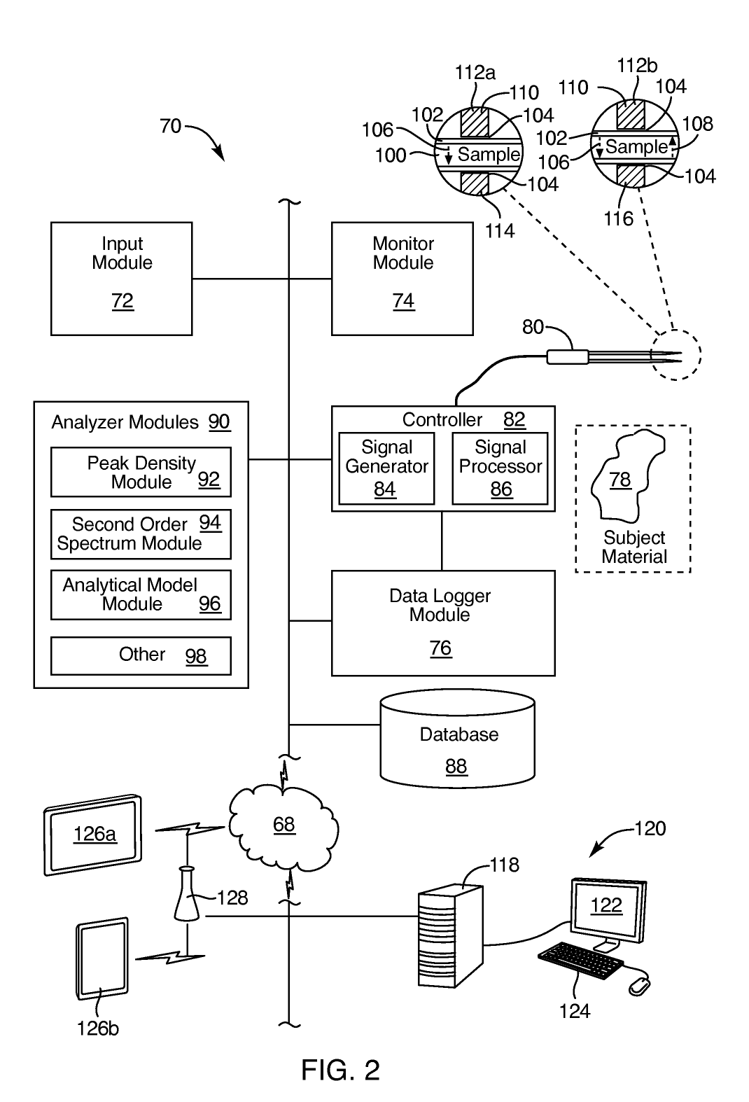

Referring to FIG. 2, an internetwork 68 may connect various components in a system 70 in accordance with the invention. In certain embodiments, all hardware may be connected by hard connections, network connections, wires, wireless connections, or over an internetwork 68 such as the internet, a wide area WIFI system, or other connection scheme. In the illustrated embodiment, input modules 72 provide inputs from data, testing, previous modeling, previous data basing, or the like. Meanwhile, a monitor module 74 (or multiple modules 74) represents, as all modules represent executables, certain executables available for monitoring operation and data from the system.

In the illustrated embodiment, a data logger 76 is responsible for logging data provided from a subject material 78 or specimen 78. Here, the specimen 78 is accessed by a probe 80. The probe 80 may be of any of the types discussed hereinabove, and hereinafter for obtaining piezoelectric or other ultrasonic transmission and reception of wave pulses. In the illustrated embodiment, a controller 82 is used to control the operation of the probe 80. Specifically, a signal generator 84 determines and provides control of the signal that will be transmitted by the probe 80 through subject material 78. Likewise, a signal processor 86 determines the analysis of the received pulse resulting from the original signal generated.

A processor 86 is responsible to perform the recognition, clean up, and other processing that may be important for cleaning up, recognizing, or otherwise providing the best possible data to be fed to the data logger module 76 for further processing. The data logger module 76 may be simplified to simply storing the data. However, according to experimental design, the data logger module 76 may control much more by way of determining what information is collected, received, and otherwise packaged into files for proper analysis.

The database 88 may be connected to the system 70 by any one of several mechanisms, including existing as a standalone system 88 having its own database engine embedded therein to control the collection, storage, management, maintenance, searching, and delivery of data stored in the database 88.

Notwithstanding the functionality of the controller 82 with its signal generator 84 and signal processor 86, as well as the functionality of the data logger module 76 and the database 88, which may include a database engine, an analyzer module 90 is responsible for the major data analysis that will be responsible to characterize tissue samples. That is, notwithstanding any signal processing or pre-processing of information in order to put it into a well structured database 88, that information must still be used on any test to perform the comparison that characterizes a tissue sample. Put another way, one ultimately useful functionality of a system 70 in accordance with the invention may be to establish instantly, in-situ, and nondestructively the differentiation of healthy tissue from malignant tissue.

Nevertheless, ex-vivo sampling may also be done. Longer and later analyses may be conducted. Also, synthetic or other materials may be distinguished. Some test other than comparison of healthy to malignant tissue may be valuable in certain circumstances. Likewise, other uses may be made of a system, apparatus, and method in accordance with the invention. Nevertheless, a critical and unserved function at present is the instant, in-situ, nondestructive, material differentiation of malignant tissue from healthy tissue.

Thus, the analyzer 90 may include a peak density module 92 for conducting peak density analysis of data. Likewise, a second-order spectrum module 94 may be responsible for conducting a second-order spectrum analysis. An analytical model module 96 may be responsible for creating analytical model analyses based on analytical models. The actual development of analytical models may be done elsewhere or may be embodied within the analytical model module 96, itself.

Meanwhile, other analysis methods may be embodied in other modules 98. For example, artificial intelligence analysis may be built into the organic (meaning built in, fully embedded, etc.) structure of any of the analysis modules 92, 94, 96, 98. On the other hand, artificial intelligence analysis may be used for other functionality such as developing templates, developing models, sorting models, determining the quality of analysis, or conducting further analysis that may require more time, or the like.

For the illustrated embodiment, the modules 90 will be described in more detail later. However, it will typically be required of the analyzer 90 to analyze data that has been collected by the probe 80. To that end, a specific sample 100 may be tested from the subject material 78 or specimen 78. The sample 100 may be a subset, and a very small subset of the specimen 78.

Here, the isolator 102 may be thought of as the layer of dielectric, impervious, protective material that separates the actual hardware of a sensor from the sample 100. Likewise, the contact enhancer 104 or ultrasonic transmission fluid 104 exists specifically to assure good ultrasonic contact that will not interfere with the readings of data.

For example, pockets of air, or other materials, but especially gases or vapors can dramatically alter the transmissivity of ultrasonic pulses. Accordingly, excellent contact provides a cleaner transmission of both the transmitted signal and the received signal. Thus, a contact enhancer 104 will typically be a liquid or gel (thixotropic fluid) that provides continuous transmission of ultrasonic wave pulses without interference or distortion. Interference and distortion may be a fact of life and contribute to the noise in any signal. Nevertheless, to the extent that they can be eliminated, the contact enhancer 104 is responsible to help do so.

A pulse 106 is transmitted and a reflection 108 may return. However, a piezoelectric element 110 may be configured as a transmitter 112, a receiver 114, or both.

Herein, trailing letters following reference numerals indicate specific instances of the item identified by the reference numeral. Thus, it is proper to speak of the numbered item by number, by number with reference numeral, or both. Thus it is not necessary to separately discuss or identify every reference letter (instance) following a reference numeral. Likewise, it is proper to speak of any or all of a numbered item, by the reference numeral alone, speaking of all in general, and of specific instances, where that is valuable for clarification.

In the illustrated embodiment, the pulse 106 may result in a reflection 108 from a reflector 116 or anvil 116. Likewise, a transmitter 112 may send a pulse 106 through the sample 100 resulting in a signal being received at the receiver 114. Here, the transmitters 112a, 112b transmit to their respective receiver 114 and reflector 116. Thus, in the instance of the transmitter 112a, the pulse 106 is a through-transmitted pulse 106. In contrast, the pulse 106 sent by the transmitter 112b passes through what is effectively the same size or distance of sample 100, but also then is reflected back from the reflector 116 as return pulse 108 in order to be received back at the piezoelectric element 110 identified as the transmitter 112b.

In operating a system 70 in accordance with the invention, individuals may work at computers 118, such as a desktop computer 118, or some other work station 120, which may include a monitor 122 of any type for receiving or interpreting information. Meanwhile, input devices 124 may enable user inputs to the system 70 through the computer 118.

Nevertheless, individuals may operate with mobile devices 126, such as the tablet 126a, PDA 126b, which may be a smart phone 126b, wirelessly accessing the system 70 through an access point 128. Wireless communication at any level and over any path may be available and appropriate. Thus, programmers operating at work stations 120 may program tests, access data from the database 88, invoke the analyzer 90, including any or all of the individual analyzer modules 92, 94, 96, 98, and so forth. Likewise, a user working through any work station 120 or other device 126, which may also provide inputs to the input module 72 (or multiple modules 72) in order to provide templates, models, data from the database 88 or elsewhere, or the like.

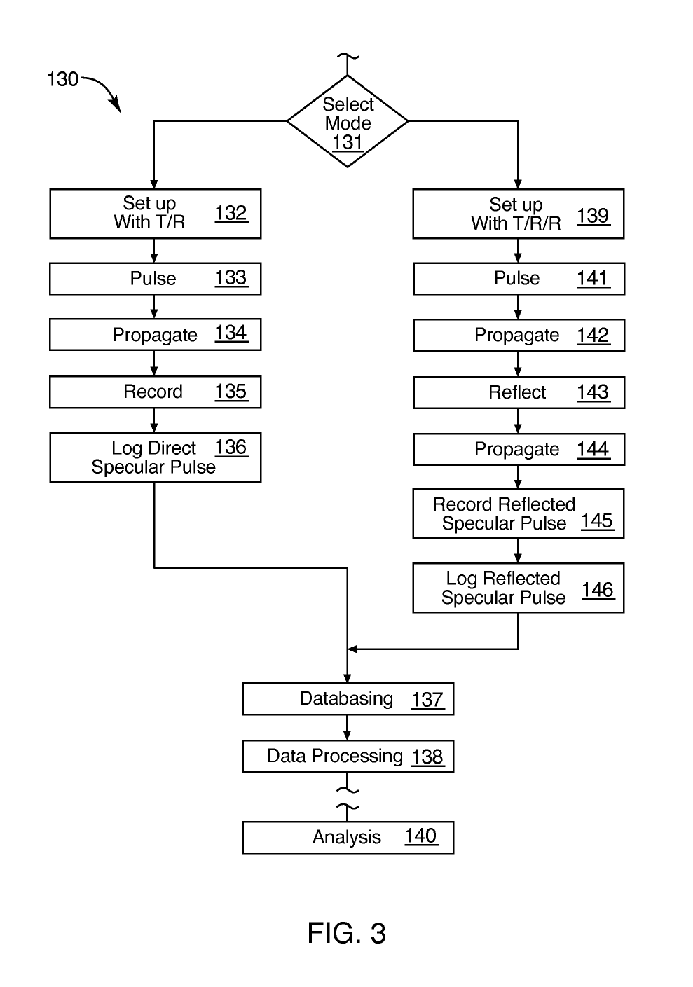

Referring to FIG. 3, while continuing to refer generally to FIGS. 1 through 22, in one embodiment of an apparatus, system, and method in accordance with the invention, a test 130 or experiment 130 may involve selecting 131 the mode of operation. For example, the decision 131 may determine whether a system will use reflected 108 or merely transmitted 106 and received through-signals.

Upon selection 131, a set up 132 may determine or may be determined by the fact that the test 131 determined that through-transmission will be relied upon. Accordingly, setting up 132 the system 70 to test and enter transmission mode will result in pulsing 133 by a transmitter 112 an ultrasonic pulse at a frequency and number of cycles suitable to propagate 134 through the sample 100. The pulse will then be detected and recorded 135.

Recording 135 may be a direct recording or detection 135. However, that information will then be logged 136 directly and will typically still be a specular pulse. That is, a specular wave may properly be referred to in terms of reflection. Specular reflection indicates that a pulse maintains its fundamental structure and carries that on, just as a mirror reflects an image.

Scattering is an actual phenomenon that will occur in the propagation of all waves in the presence of materials that may alter those waves in direction, velocity, absorption, or the like. In the illustrated embodiment, the through-transmission methodology generates a specular pulse 133, which is propagated 134 through the sample 100, and then detected 135 or recorded 135 by the receiver 114. In contrast, in the other mode to which the test 130 may turn, a reflection will require a setup 139 of a pulse to be transmitted, reflected, and then received.

After setting up 139 the transmitter 112b, a reflector 116, and the sample 100, a pulse 141 is generated or pulsing 141 occurs by the transmitter 112b generating a wave propagated 142 through the sample 100. Reflecting 143 off the reflector 116 (anvil 116), a return or reflected wave 108 is propagated 144 back through the sample 100 toward the sensor 112b that acts as the detector for detecting 145 or recording 145 the specular pulse received.

Again, logging 146 that reflected pulse rather than a directly transmitted pulse results in databasing 137 the information from the test 130. Other data processing 138 including analysis by the analyzer 90 by any of the processes 92, 94, 96, 98, or the like may then occur.

As a practical matter, analysis 140 need not follow directly nor immediately. Typically, data processing 138 will be required of all signals, and may be embodied in the analysis 140, or replace the analysis 140. Nevertheless, the analysis module 140 is shown in one embodiment as occurring at some different point in space or time from the initial data processing 138.

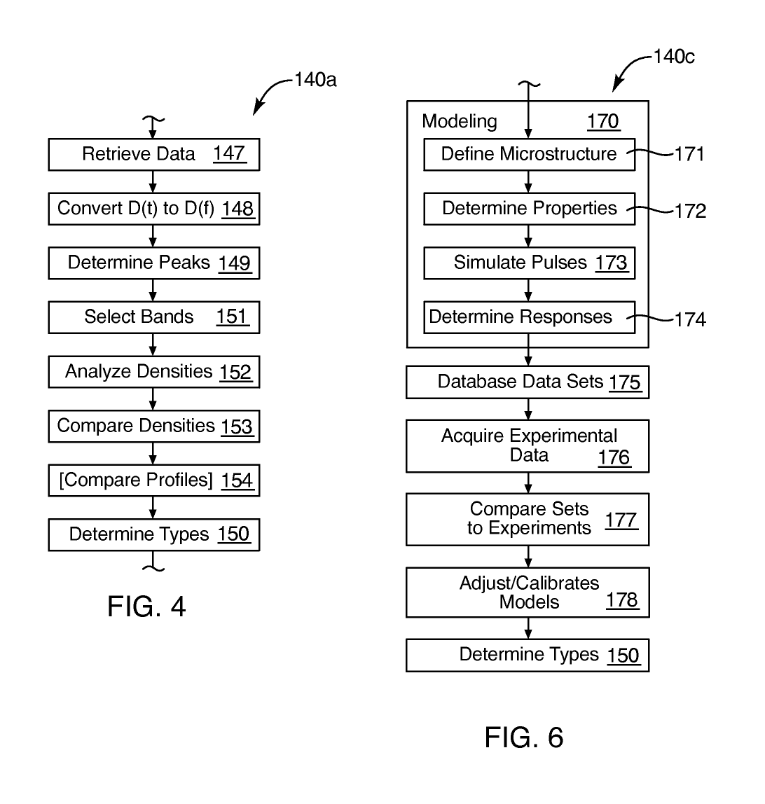

Referring to FIG. 4, a process 140a for peak density analysis may begin with retrieving 147 data, such as from the database 137. In reality, retrieval of data directly from the probe 80 is a possibility. However, as a practical matter, data may be gathered in great volume, and with a very questionable signal-to-noise ratio. Thus, post processing may be required, sufficient quantities may be required to be collected through data basing 137, before a statistically significant sample can be relied upon, or the like.

By one mechanism or another, retrieving 137 data may begin in order to provide data to be converted 148 from a time domain to a frequency domain. Determining 149 the peaks, and the location of each, will not necessarily require that the frequency of all the peaks be determined, nor even the frequency of any peak.

In peak density analysis 140a, determining 149 the peaks may be a matter of determining where the peaks are, and then selecting 151 the bands in which the peaks will be analyzed. Analyzing 152 the densities includes determining how many peaks are within certain bands 151 of interest. As data is collected and databased 137 over long periods of time, multiple samples, multiple patients, and so forth, the databases 88 available will become greater. The bands selected 151 will be more defined, well understood, well known, and so forth. Thus, if a malignancy is known to be of a particular type, then selecting 151 in bands where those corresponding peaks will be expected or be uniquely characteristic may be selected 151.

Thus analyzing 152 the densities within the selected 151 bands may then result in comparing 153 the known densities in the known bands with templates, previous data, or other analysis available. Comparing 154 profiles may also be done. However, this is indicated in brackets indicating that it is optional. Comparing 153 the densities in specific bands may provide an immediate result.

However, comparing 154 profiles such as numbers and the frequencies at which those particular numbers occur, may be found useful. Ultimately, determining 150 the types involves the analysis or decision that must be made as to whether a particular sample 100 is of a particular type of material, typically a class of marginal tissue sample 100 in this case.

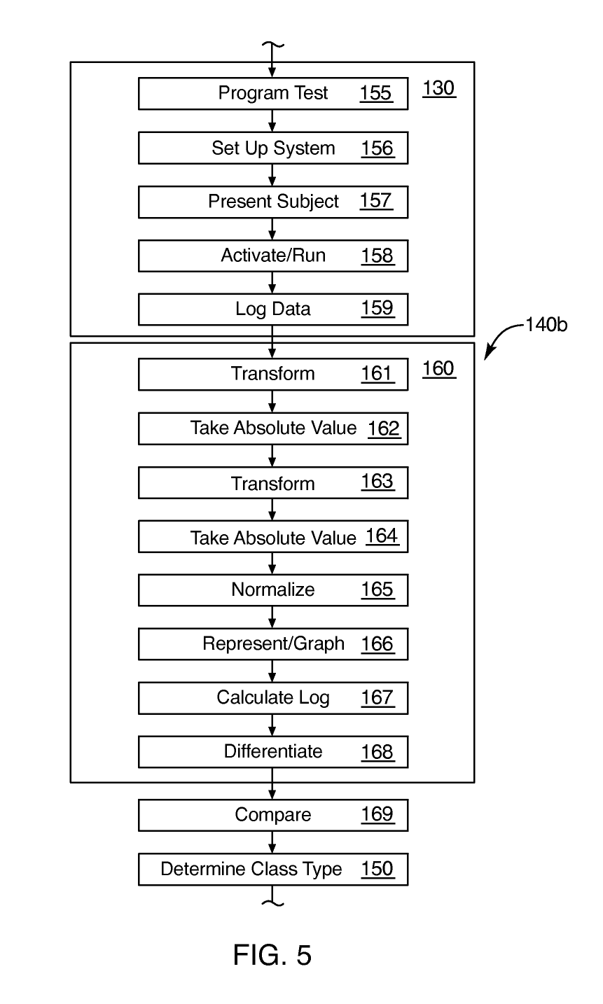

Referring to FIG. 5, while continuing to refer generally to FIGS. 1 through 22, a method 140b for the second-order spectrum analysis may be built into a process that begins with the test 130 or experiment 130 that has been programmed 155 and set up 156 as discussed hereinabove. Presenting 157 a subject 100 or sample 100 may involve presentation 152 of the subject material 78 or the general tissue sample 78, from which a very small sample 100 is actually touched or tested by a system 70 in accordance with the invention. Activating 158 the system 70 results in logging 159 data that may be represented by a series of curves.

The process 140b described herein as the second-order spectrum analysis involves transforming 161, typically by a Fourier transform, the original raw data. Thereafter, taking 162 an absolute value of that data, one generates a different curve representation in a power spectrum, rather than a time domain. Thereafter, transforming 163, with another Fourier transform, one may take 164 another absolute value. That absolute value taken 164 is then normalized 165, typically by taking the maximum value within the test or some other maximum, and dividing all values by it. This presents yet another curve that has been normalized 165, such that it may be characterized and compared generically by its relative magnitude, rather than its absolute magnitude.

Representing 166 this curve or graphing 166 this curve then permits, whether numerically, digitally, by analog methods, or the like, calculation 167 of the logarithm, to the base 10 in one presently contemplated embodiment, of the graph 166. One may then differentiate 168 based on the log calculated 167 for the graph 166. One may now compare 169 the sample against other templates, experience, and the like, thus determining 150 the class of the sample 100. Here, in finding the tissue types at margins of surgery, one may determine 150 the type of tissue, whether clean, healthy, or malignant, and so forth.

Referring to FIG. 6, in one embodiment of a model-based data analysis 140c, a modeling process 170 may include defining 171 microstructure of tissue. This may include identification of any component and mechanical property of a tissue sample, cell, molecule, protein, or the like of interest, in terms of its effect on ultrasonic waves.

For example, the particular phenotype of a cell or tissue may be identifiable by its cell size, nucleus size, tissue microstructure, bulk modulus, shear modulus, elastic modulus, other cellular or extra cellular components, and the like. Thus, one may characterize the microstructure in terms of component, size, shape, and material properties in order to model its behavior.

Thereafter, determining 172 the properties may be a matter of chemical or mechanical analysis. Thus, determining 172 properties may be a matter of experimentation, documentation, research, identification of information available from microscopic investigations, or the like. The purpose of determining 172 the properties associated with each identifiable element of the microstructure defined 171 for a sample material (e.g., tissue, cell, etc.) is to determine the mechanical characteristics that will affect ultrasonic transmission, reflection, scattering, absorption, and so forth.

Having defined the sizes and properties of a tissue or cell, one may simulate 173 pulses, propagating them through the model of the microstructure. Accordingly, responses of the pulses to the microstructure will allow an analytical determination 174 of the response to the pulse. Thus, whether the ultrasound is modeled as a through-transmitted pulse 106 or as a reflected pulse 108, the computerized model may determine 174 the behavior of the combination of the tissue sample 100, and the propagated pulse 106.

At this point, databasing 175 data sets from various configurations may be done just as experimentally, and saved in the database 88 identifiable as whatever the data represents. Similarly, acquiring 176 experimental data may also be valuable both for prediction, as well as verification of modeling. Comparing 177 data sets from the modeled outputs of the modeling process 170, allows comparison 177 of experimental data to modeled data. If the data is sufficiently robust, clean, and clear, one may be able to use it directly for determining 150 the types or phenotypes in question. Thereafter, adjusting 178 or calibrating 178 the models may be done.

However, in other embodiments, adjusting 178 or calibrating 178 the models may occur by the comparing 177 of the model sets of data to experimental sets of data. At that point, the model may be improved sufficiently by correcting for nuances that may not be modeled sufficiently precisely nor understood well enough to model.

Engineers often refer to “empirical” adjustments. With a little less respect, people refer to “fudge factors” or “offsets,” and the like. These simply represent the adjustment that must be made to a purely theoretical model, which may be somewhat limited in its detail, to adjust for other influences that may be too fine, undetectable, or complex to be modeled. As an engineering procedure, such calibration 178 or adjustment 178 to fit or adjust a model to better predict results consistent with experimental data is a fact of technical life.

Ultimately, all models may eventually be adjusted 178 or calibrated 178 over time in order to improve the determinations 150 made by applying the model. Thus, determining 150 the types of cells, or the phenotypes of tissues themselves, may be done with increasing accuracy over time, as the database 88 grows.

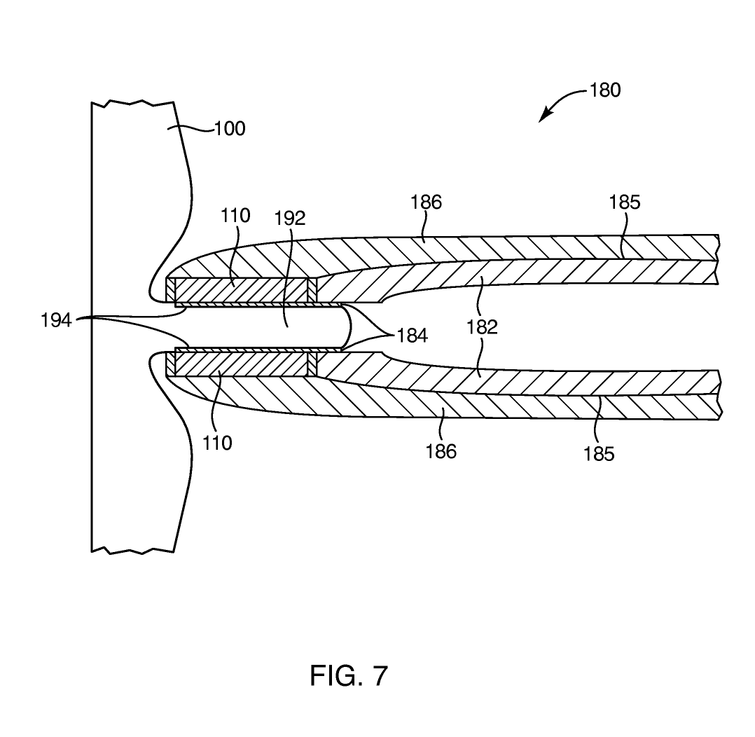

Referring to FIG. 7, a device 180 for determining the pathology of biological tissue, whether excised (ex-vivo) or in vivo may be configured as a set of forceps 180. The forceps 180 may include a conductor 182 and a contact 184. In reality, the contact 184 may only be necessary in the illustrated configuration because the conductor 182 may be constrained mechanically from extending over the piezoelectric element 110. Here, a contact 184 provides for a planar region that will establish the test portion or test region within the forceps 180.

Meanwhile, an insulator 185 may separate one conductor 182 or an inner conductor 182 from an outer conductor 186. Here, the conductors 182, 186 need not constitute the entire mechanical portion of the forceps 180. However, as illustrated, multiple conductors 182, 186 provide electricity across the opposite faces of the piezoelectric transducers 110.

A specimen 100 or sample 100 is thus flanked by the transducers 110. A portion 192 thereof becomes the intermediate material between the transducers 110 that will propagate the ultrasonic pulse.

In the illustrated embodiment, a film 194, such as a coating, or a separate material layer, may be available for insulating or otherwise protecting against chemical action, electrical conductivity, or the like. In this embodiment, the forceps 180 may determine the pathology of a comparatively small-volume sample 192 in vivo. Of course, ex-vivo samples would also be possible. However, one benefit of an apparatus and method in accordance with the invention is the ability to do a rapid and accurate test in vivo of margins of surgery.

Here, high-frequency ultrasonic pulses in the range of from about 10 to about 100 megahertz may be sensitive to a range of pathologies in tissue including fibroadenomas, fibrocystic changes, atypical ductal hyperplasia, ductal carcinoma, and lobular carcinoma. High-frequency ultrasound appears to be sensitive to molecular subtypes of cancer cells based on their biomechanical properties, ultrasonic scattering characteristics, and so forth. Such results have been obtained from through-transmission measurements, where the transmitted pulse is analyzed directly. They have been taken also as pulse-echo measurements from a specular surface where the reflected pulse is analyzed directly. This stands in contrast to conventional ultrasonic characterization methods. Those rely on diffuse scattering of non-specular ultrasonic signals. Such scattering may arise from macroscopic or microscopic structures in cells of tissues. The instant devices and methods use a different analysis technique. They use, as well, a different ultrasonic frequency range and a different ultrasonic pulse application.

Here, the apparatus 180 employs direct through-transmission of propagated pulses 106, as well as pulse-echo (pulse 106 and echo 108 or reflection 108 thereof). Optimal signals appear to be in the range of from about 10 to about 100 megahertz. Accordingly, the piezoelectric elements 110 may be comparatively thin.

For example, for a 50 megahertz transducer, the approximate thickness is about 50 microns. Because these electric elements 110 are so thin, they may easily be manufactured in high-frequency, ultrasonic, micro-sensors. Thin film deposition techniques, including sputter coating, plasma-enhanced chemical vapor deposition, ion beam-enhanced deposition, and molecular beam epitaxy are all suitable.

Moreover, incorporating these micro-sensors 110 into the tips of the forceps 180 or other device 80 allows the micro-sensors 110 to analyze a comparatively much smaller amount 192 of tissue held between the tips of the forceps 180. This configuration permits the introduction of ultrasonic sensing capability into regions that are simply not available to prior art devices. For example, a surgical cavity along the alimentary canal, lung tissue, or the tip of a biopsy needle is sufficient for access by such a system 180.

The ultrasonic signals acquired from the device 180 may be analyzed in any of the ways discussed hereinabove, and detailed hereinbelow. Ultrasonic wave speed, variations thereof, wave attenuation, and spectral features such as the peak density (number of peaks and valleys in a specified frequency band of the ultrasonic spectrum) are all available. Moreover, other analysis may be applied to the data.

For example, characterization of spectral peak shape with a feature recognition program may be done as is done in principal component analysis (PCA). In fact, in one embodiment, a pattern recognition algorithm such as that introduced by Cook in U.S. Pat. No. 6,546,378, issued Apr. 8, 2003, entitled SIGNAL INTERPRETATION ENGINE; U.S. Pat. No. 6,745,156, issued Jun. 1, 2004, entitled SIGNAL INTERPRETATION ENGINE; U.S. Pat. No. 6,988,056, issued Nov. 4, 2004, entitled SIGNAL INTERPRETATION ENGINE, provides a methodology for characterizing unknown features, simply by signal processing and artificial intelligence. Likewise, U.S. Pat. No. 6,804,661, issued Oct. 12, 2004, to Cook and entitled DRUG PROFILING APPARATUS AND METHOD also provides practical applications of such artificial intelligence engines. Such engines may be applied to characterizations of tissue samples in an apparatus and method in accordance with the invention.

The forceps 180 may be manufactured in numerous other ways. However, so long as means exist to conduct electrical signals to and from the micro-sensors 110, either through the metal components 182, 186 of the forceps 180, or through lines secured therein or thereto, the electrical connections may be made. Similarly, some mechanism 185 to isolate the conductive paths may be noted and accomplished as illustrated, or accomplished by insulation deposited directly onto leads embedded in or otherwise attached to the forceps 180.

The application of a thin, conductive electrode coating one side of the piezoelectric element 110 is required because the piezoelectric effect is the distortion in response to electrical voltage, and the electrical voltage output in response to a distortion. Thus, piezoelectric devices 110 are reciprocal in their behavior.

In certain embodiments, an insulating film 194 may be applied by any number of mechanisms, and may be a polymer, a ceramic, or the like. For example, aluminum oxide, silicon dioxide, and the like may be suitable, as may numerous polymers including polypropylene (PP), polysulfone, polytetrafluoroethylene (PTFE), and others that are capable of comparatively high temperatures required by autoclaves for such instruments' sterilization.

A gauge measuring pressure applied to the sample 192 grasped between the piezoelectric elements 110 of the forceps 180 may be automated in order to control and measure forces, pressures, and the like. Similarly, a locking mechanism may be effective if handles are manually operated. Thus staying in control of automated handles may also serve the same function. Distances are measured by meters which may be implemented in numerous ways known in the art, including proximity sensors, direct measurement devices, and the like.

Optical sensors may also be created that measure within fractions of a wavelength of a particular wavelength of light transmitted between separated members defining the thickness of the sample 192 or sample region 192. Thus, engineering metrics principles may be applied in order to get resolutions on the order of about 0.1 micron. Similarly, fiberoptic thickness gauges may also be used. The piezo-electric sensors may yield data to be processed to determine distances. Such technologies are available commercially and need not be identified in detail herein.

Thus, in summary, a device 180 in accordance with the invention provides unique incorporation of a high-frequency ultrasonic micro-sensors 110 into forceps 180. Likewise, the use of direct through-transmission as well as specular pulse-echo measurements is available on the pulses 106, 108.

The data analysis modules of the analyzer 90, including the peak density module 92, the second-order spectrum 94, the analytical modeling module 96, and other artificial intelligence engines or other units also provide analysis of signals not relied upon the prior art. The use of specular pulses and waves rather than diffuse reflected measurements provides a resolution capable of characterizing features at a microscopic level. Likewise, the various data analysis programs may include combinations thereof.

Thus, comparatively higher resolution (on the order of less than a millimeter) determination of pathology and tissue specimens in a non-laboratory setting provides much more speed. Thus, even an operating room, clinic, field hospital, or a non clinical setting such as a region that has no technical laboratory facilities may be served. This condition may exist either because it is underdeveloped or because of the existence of a natural disaster. In any event a practitioner with a tool 180 may have access to such analysis online, immediately, and reliably. Likewise, an apparatus 70, 180 and methods 130, 140 in accordance with the invention provide rapid sensing of pathology for guiding surgery, biopsies, and endoscopic procedures in situ and simultaneously, rather than post operatively.

The apparatus 180 may probe multiple tissue regions in vivo or ex-vivo rapidly allowing mapping of a tissue pathology in a critical region. Thus, at a surgical margin, where removal of all malignant tissue is vital for reducing recurrence of a disease, an apparatus 180 in accordance with the invention may probe for metastases in tissue regions surrounding a primary tumor. This may be done immediately, during surgery, even by the surgeon as he or she seeks to establish definitively the margins of non-malignant tissue.

It is not unnoticed nor unuseful that minimal invasion is required to take tissue samples. Tissue need not be removed, severed, or even damaged by the sensors 110 of the apparatus 180. Likewise, the small size allows incorporation into biopsies, endoscopic probes, or intra-operative use inside small surgical cavities.

In certain embodiments, an apparatus 180 in accordance with the invention may be used for real-time, intra-operative assessment of surgical margins during cancer surgery, to ensure all malignant tissue has been removed after excision of the tumor. This may be done in excised tissue, in vivo tissue, or both. For example, in obtaining cancer-free margins in breast conservation surgery (lumpectomy), brain cancer surgery, lung cancer surgery, prostate cancer surgery, and Mohs surgery for melanoma, such a system can be invaluable.

Likewise, incorporated into an endoscope, an apparatus 180 may be used for minimally invasive biopsy of malignant lesions in vivo with real-time pathology results, more accurately than current abilities to determine the extent of the malignancy. Examples of this include colon cancer, esophageal cancer, stomach cancer, bronchial cancer, and throat cancer.

Even a biopsy needle may have incorporated into it a system such as the forceps 180 extending to the tip of the needle at multiple depths as the needle is being inserted into the tissue. Likewise, tissue removed in a biopsy may be tested at each step without the need to remove the tissue or analyze it separately with conventional pathology. Moreover, such a system can profile the depth to determine the extent of a pathology, and even its rate of change.

In certain embodiments, a device 180 in accordance with the invention may be used externally or semi-externally for rapid screening of skin and oral cancer. Incorporation into an intravenous catheter provides a device 180 to determine the pathology of tissues in many parts of the body including arterial walls, heart, brain, and so forth.

The nature of operation of the apparatus 180 ensures that no tissue need be damaged, or even excessively disturbed, let alone removed. The apparatus 180 may therefore provide a mechanism for testing various regions of the brain or other neural systems in the body for neurodegenerative diseases.

Physicians in non-clinical environments, lesser developed countries, and other environments lacking the time, cost, space, or the like may still receive cost-effective pathology results from very small tissue samples.

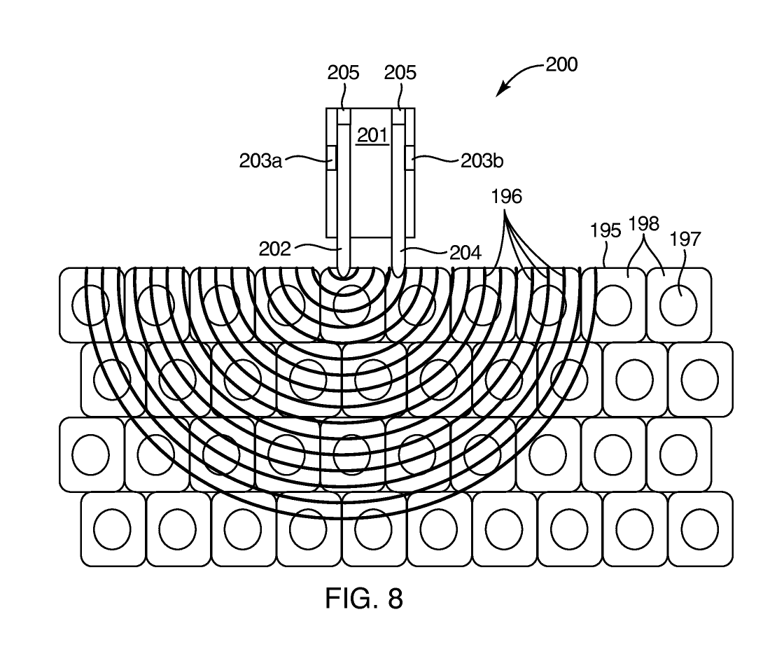

Referring to FIG. 8, in one embodiment of an apparatus and method in accordance with the invention, a wave 196 may be propagated through cells 198, and particularly along the surface thereof. In such an apparatus and method, a probe 200 may include a housing 201 providing support 201 to a transmitter probe 202 and a receiver probe 204. In the illustrated embodiment, the apparatus 200 sends with a transmitter 200 a wave traveling along the surface of the cell structure 198, and detected by the receiver probe 204.

The properties of the cellular materials 198 affect the transmission of the wave 196 or pulses 196. Thus, for example, one may characterize different cells 198 or cellular structures 198 in terms of density, stiffness, and the like. One may solve for the modes of resonance and transmission in free space, and couple those to the medium as it alters how the transmission occurs. Likewise, samples provide a medium for influence on the wave propagation between the rods 202, 204.

Thus, sending with one rod 202 or probe 202 to another probe 204, constitutes the overall probe system 200. These probes 202, 204 need not permanently penetrate, but may penetrate not at all, or some distance with minimally invasive effect. Transducers 110 may exist at the base of the probes 202, 204, and thus cause the rods 202, 204 or probes 202, 204 to vibrate at ultrasonic speeds.

In the illustrated embodiment, detected biological tissue properties at the microscopic level include histopathology, molecular pathology, or fine tissue incongruities, such as cancer margins and the borders between various tissue types. Surgery and excised tissue samples are possible. The two needle-shaped probes 202, 204 positioned at the end of a pen-like instrument 201 or housing 201, together with the probes 202, 204 and transducers 110 form the basis of the instrument 200.

One probe 202 is a transmitter and the other 204 is a receiver. The probes are simultaneously placed against the tissue surface to collect ultrasonic measurements. The transmitter 202 emits a high-frequency (e.g., 10-100 megahertz) ultrasonic waveform pulse. The waveform pulse is picked up by the receiver 204 so that it may be compared with the transmitted waveform.

A resulting signal may be analyzed, as described hereinabove, based on principal components thereof or otherwise, against the database 88 of other empirically obtained waveforms. Likewise, it may be compared against computationally modeled waveforms as described hereinabove. Thus, based on various historical samples from an empirical test or from models, various tissue properties such as bulk modulus, cell size, and the like may be accommodated, and thus provided for in an analysis as described hereinabove. The other data analysis methods described herein may also be applied.

In certain embodiments, pressure sensors may be incorporated into either the probes 202, 204 or the housing 201 in order to measure and compensate for tissue deformation induced by the pressure applied by the device 200.

Thus, the apparatus 200 includes a transmitting transducer 202, a receiving transducer 204, and may include a pressure detector. The pressure detector may be embodied in the housing 201. In fact, the sensors 203a, 203b may be connected to the probes 202, 204, respectively to actually serve as the piezoelectric elements 110. Likewise, sensors 205 may, by the same mechanism or other mechanism, detect the pressure imposed on or by the probes 202, 204, which must be accommodated in an analysis.

The piezoelectric material 203 may be directly embedded or fabricated into the probes 202, 204. However, it also may be associated with the outside of the electrodes. Electrical connections corresponding to those of FIG. 7 may be embodied in the apparatus 200. A metal, plastic, or ceramic material configured into the needle-shaped probes 202, 204 attached to a piezoelectric transducer 203 may contact the surface of a tissue 198. The probes 202, 204 may actually operate as acoustic wave guides, providing paths to transmit the ultrasonic pulses to and from the piezoelectric transducers 203. Thus, numerous embodiments may be configured based on the same operational principles.

Illustrated, schematically herein are not only the cell structures, but nuclei 197 thereof, characterizing the microstructures of the cells 198. In operation, the probes 202, 204 are in simultaneous contact with the cells 198, or tissue 198 in order to take the measurement.

Ultrasonic analysis methods may rely on a device 200 tuned to a range of frequencies sufficiently high to respond to small (even the smallest significant) cellular structures and properties required to detect differentials or differentiations between key cell types. For example, bulk modulus is influenced by cytoskeletal aberrations indicative of a range of disease states. Likewise, in certain embodiments, comparatively short duration, high-frequency (HF) pulse-echo measurements are transmitted from one transducer 203 and probe 202 reflected from the first few cell layers, and received back by the second high-frequency transducer 203b.

In another embodiment, surface longitudinal waves 196 are generated on the surface of the tissue by one transducer 203a through its corresponding probe 202 and propagated across the tissue surface 195 of the cells 198. There, it is detected by the second high-frequency transducer 203b through its probe 204. The surface longitudinal wave 196 is typically constituted by tissue vibrations that will penetrate several cell layers into the tissue 198, thereby providing an adequate sampling of the near surface tissue to an approximate depth of from about 30 to 100 microns.

Received signals may be amplified, filtered by a high-pass filter, or otherwise conditioned in order to improve the signal-to-noise ratio. They may be digitized, recorded, and converted to their particular spectra. Spectra may then be analyzed with principal component analysis (PCA) in conjunction with waveforms modeled on algorithms based on various cell properties.

The waveforms can also be converted to an auditory signal that can be used as a comparison method to identify to a practitioner tissue abnormalities, incongruities, or other differences that may be difficult to detect by visual or other inspection.

In the illustrated embodiment, whether medical, veterinary, or research, the apparatus 200 may provide generation of a high-frequency, surface-longitudinal wave that characterizes and determines properties in the near-surface cells 198 or tissues 198. The apparatus 200 makes generation and receipt of such a surface wave 196 possible by placing the sender 202 and receiver 204 close to one another. These may even be close on a microscopic scale, on the order of from about 0.2 to about 2.0 millimeters. Prior art devices do not operate on this principal or in these ranges.

Moreover, the transmitter 202 and receiver 204, which actually are probes 202, 204 between the transmitter 203a and receiver 203b, are sufficiently close to detect fine margins of a few to several cells 198. Thus, surgeons may “hear” cell size, bulk modulus, and other properties not easily seen by the naked eye. Thus, a surgeon may advance in excising a tumor by having more information (and more reliably) than is available by conventional pathologies in a laboratory far removed in time and space from an operating room.

An apparatus 200 in accordance with the invention is sufficiently versatile to be used in medical diagnosis of disease where tissue and cellular incongruities have a characteristic ultrasonic signature pattern that can be identified thereby. Detecting a histopathology of many cancers is possible due to their inherent consequences that change tissue structures.

For example, molecular subtypes of cancers may result in over expression or under expression of certain cytoskeletal regulatory proteins. Similarly, neurological disorders are sometimes accompanied by a breakdown of cytoskeletal components, intercellular connected proteins, and the like. These affect mechanical properties of cellular components. Those properties affect ultrasonic propagation and reflection.

In surgery, the apparatus 200 may distinguish between types of tissue by connection to a dual-color or tri-color display 122 or monitor 122. This may guide with a colored imaging approach, to “see” properties that are otherwise visually undetectable.

Likewise, an audible tone may report by dual-tone or triple-tone auditory processors to distinguish between various known types of tissues. This type of an interface may be valuable to a surgeon in finding the margins of a tumor intraoperatively (during the actual operation). Likewise, such a system may be used in defining margins of tissue breakdown or inflammation as in the case of infection, autoimmune response, toxic reaction, or the like.

Quite opposite a medical context, agriculture and the food industry, and particularly the meat and poultry industries, have ongoing quality control requirements imposed by regulation, consumer demands, and so forth. In one embodiment of an apparatus and method in accordance with the invention, the identification of tissue type, tissue density, percentage of fat, possible disease or illness, and the like may be taken by an apparatus and method in accordance with the invention. Thus, detection or gradation of quality parameters may be served by such a system 200.

In one presently contemplated embodiment, such a system 200 may be used for detecting the quality control parameter on tenderizing or processing of meats characterizing homogeneity, analyzing protein content, identifying percentages of various tissue types, and so forth.

Likewise, mapping may also be done. Protein content and other pathological indicators may be modeled and tested. A computer may train the sensors, recognition programs to identify pathological indicators, cell size, densities, pathologies, or other characteristics.

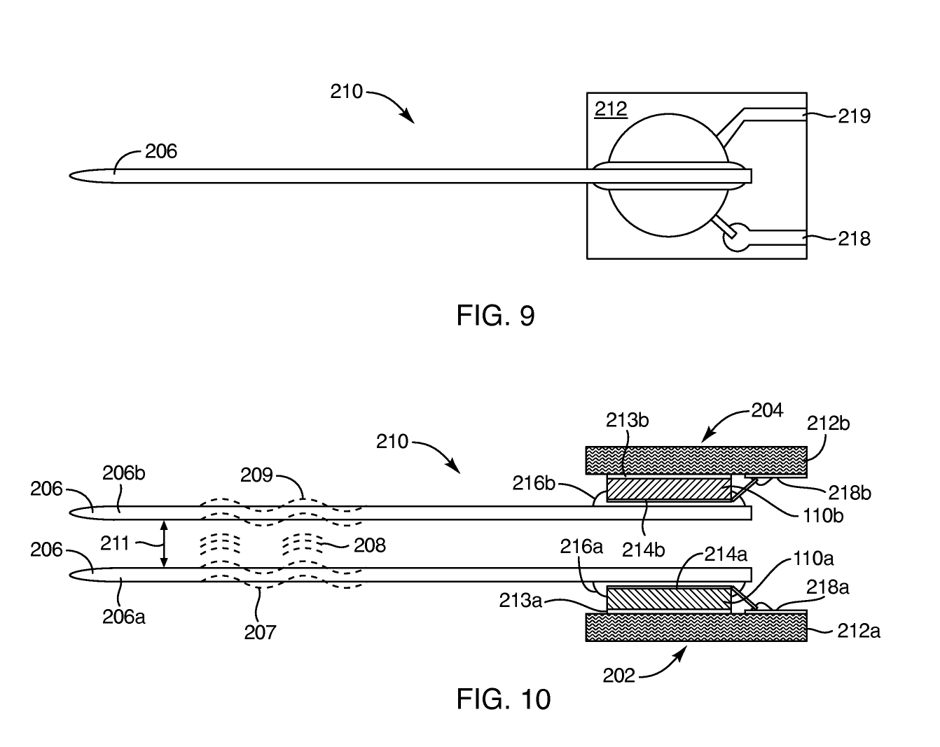

Referring to FIGS. 9 through 11, an apparatus and method in accordance with the invention may rely on micro-needles 206a, 206b imposing ultrasonic vibrations 207 by means of a pulse 208 detected as a vibration 209 by the micro-needle 206b of the apparatus 210 or micro-needle system 210. In the illustrated embodiment, micro-needles 206a, 206b are separated by a distance 211 or gap 211. Across that gap 211 is propagated the vibration 207 or wave 207 as a pulse 208 of some duration, as described hereinabove.

Across the gap 211 or distance 211 in the subject material 78 (of specimen 100 or sample 100), the pulse 208 travels. It is detected as the vibration 209 or oscillation 209 in the receiving micro-needle 206b. In the illustrated embodiment, a base 212 may be constituted by components 212a, 212b, or a housing 212 that effectively becomes the base 212, or the like.

Electrodes 213a, 213b provide a voltage across the piezoelectric element 110a, 110b in order to impose the piezoelectric effect on the piezoelectric transducer 110. Again, a voltage across the piezoelectric device 110 imposes distortion or movement. A waveform applied as a voltage to a piezoelectric device 110 causes mechanical movement (oscillation) of the surface of the piezoelectric element 110.

Thus, the piezoelectric element 110a becomes an ultrasonic sender or transmitter by imposing a vibration on the micro-needle 206a. Meanwhile, the piezoelectric element 110b or transducer 110b operates as a receiver 110b receiving mechanical oscillations imposed as vibrations 209 on the receiving micro-needle 206b.

Typically, infrastructure such as bonding 216a, 216b may secure the piezoelectric elements 110a, 110b to their respective portions of the base 212. Leads 218a, 218b may connect to the front face or the portion of the piezoelectric elements 110a, 110b, respectively that connect to the micro-needles 206, 206b, respectively. As illustrated, an electrical connection is also made to the opposite faces of the piezoelectric transducers 110a, 110b.

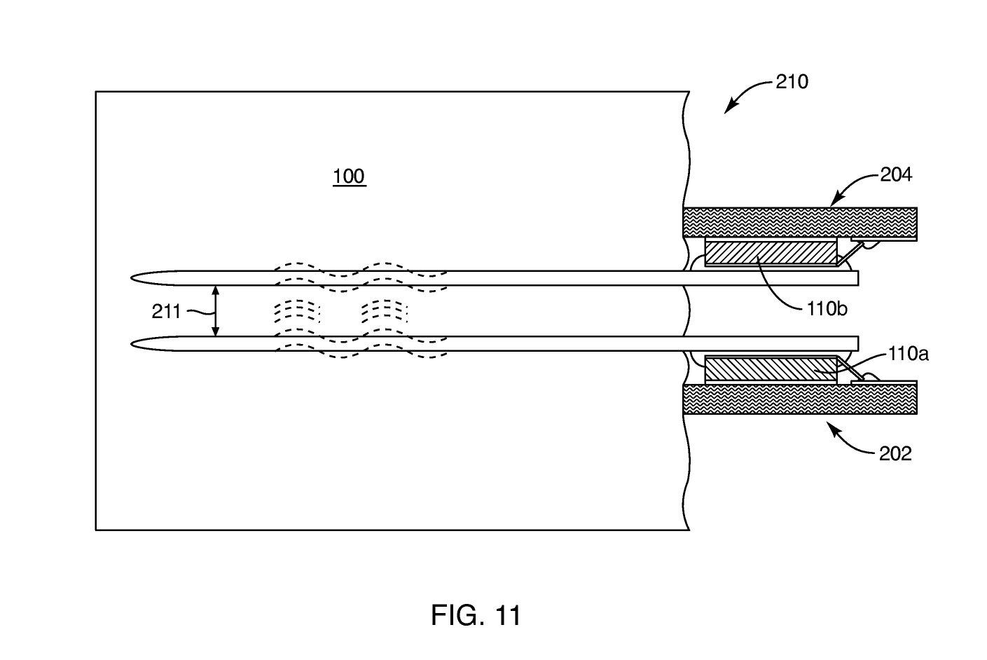

Referring to FIG. 11, operation of the system 210 is still quite minimally invasive. Notwithstanding the needles 206 are actually penetrated into the cell structure of a specimen 100, they do no more damage and leave no more consequences than a hypodermic needle. In operation, such a system 210 may rely on the twin needles 206 as probes 206 penetrating a biological material, such as tissue 198 and imposing thereon a high-frequency (HF) ultrasonic signal. The frequency range is high enough to respond to microstructures within the biological material.

Again, one needle 206a acts as a transmitter, while the other 206b acts as a receiver for through-transmission measurements. Alternatively, one needle 206a may be used as a transmitter-receiver sensor 206a while the other acts as a reflection surface 206b for pulse-echo type measurements. Again, as in previous apparatus, time domain and frequency domain waveforms may thus respond or react to the properties of microstructures of the material into which the micro-needles 206 are embedded. The responses may be analyzed, compared, or otherwise evaluated in view of previous experimental waveforms, model waveforms, or the like as described hereinabove.

A system 210 may thus determine pathology, composition, characterizations, and the like of cells, tissues, and so forth. Again, whether ex-vivo excisions or in vivo live tissues, the apparatus 210 may operate similarly. Again, as of the apparatus of FIG. 8, other materials, including food products such as meat, and the like may be characterized. The information about a genetic expression signature is indicative of tissue types, tissue structures, and pathologies or other information that may also be revealed.

Typically, the micro-needles 206 function as acoustic wave guides for propagating ultrasonic waves between the piezoelectric sensors 110a, 110b and the biological material specimen 100. The vibrations 207 or displacements 207 are generated by the oscillation of the voltages on the piezoelectric elements 110a, 110b to produce the ultrasonic pulses 208 transmitted to the receiving probe 206b or needle 206b.

Typically, the base 212 is dielectric and does not conduct electricity. It also may be used as a mechanical base in order to comparatively rigidly support the ultrasonic piezoelectric elements 110 in order to assure the vibrations 207 in the needles 206. The bonding agent 216a, 216b may be of any suitable type, polymeric, epoxy, or the like suitable for maintaining a solid, even rigid, connection capable of transmitting the piezoelectric signal from the piezoelectric elements 110 to the needles 206 with minimal absorption or attenuation thereof.

In use, the biological sample 100 may surround the needles 206 thereby providing a path between the needles 206 propagating cleanly the pulses 208. Again, the applications described with respect to FIG. 8 may apply hereto. The differences and the abilities with respect to the same pathologies, tissue types, characterizations, and the like apply here likewise. Again, the adaptability to both through-transmission and reflected transmission are available.

With respect to the structure, an apparatus and method in accordance with the invention may be employed in a broad array of applications. Moreover, by producing signals in the 10 to 100 megahertz (high frequency) range, the piezoelectric elements 110 may be comparatively thin, on the order of about 50 microns for a 50 megahertz transducer 110.

Again, because the elements are sufficiently thin, manufacturing may include film deposition technology such as sputter coating, plasma-enhanced chemical vapor deposition, ion beam-enhanced deposition, and molecular beam epitaxy. The micro-sensors 110a, 110b may be also be incorporated into the needles 106a, 106b also. Such a configuration may provide for mass-produced, assembly-line facilities reducing costs substantially.

Here again, the incorporation of high-frequency, ultrasonic micro-sensors 110a, 110b into two parallel, miniature needles 206, 206b for direct, through-transmission as well as pulse-echo measurements relies on the needles 206 as acoustic wave guides. Through-transmission as well as specular, pulse-echo measurements stand in contrast to conventional ultrasonic systems with their diffuse reflection measurements.

Likewise, the data analysis programs discussed herein assist in determining the microstructure of the biological material sample 100 and identify tissue pathologies from the signals received and processed. Associated instrumentation as discussed with respect to other embodiments described herein and in the references incorporated by reference herein provide for a compact, portable, robust system that may conduct tests, read and analyze the resulting signals, and provide unique determinations with a minimum of supporting structure, and no laboratory required offsite.

An apparatus 210 in accordance with the invention may provide high resolution (less than 1 millimeter) determination of pathology and tissue specimens in a non-laboratory setting. As discussed with respect to alternative embodiments hereinabove within an operating room, clinic, field hospital, non-clinical setting, location with a lack of technical facilities or laboratories, a developing or undeveloped country, or in a disaster situation with an absence of such equipment, such a device 210 may serve well. It may operate to provide the necessary differentiations, identification of pathologies, identification of tissue structures, and the like.

Likewise, intraoperational surgeries and endoscopic procedures may also be conducted. Again, the ability to probe multiple tissue regions in vivo or ex-vivo in rapid succession with immediate analysis allows mapping of tissue pathology. It may include not only the tissue conditions or the tissue pathology at a particular location, but the change and even the rate of change throughout a region being tested.

In general, the miniature needles 206 need only have a length of from about 3 to about 5 millimeters and a separation of from about 1 to about 2 millimeters. Thus, such a device is minimally invasive and is even much less invasive than a hypodermic needle.

Thus, in general the applications, uses, and so forth applicable to the apparatus and methods corresponding to FIG. 8 hereinabove may also be applied. The types of infections, malignancies, and the like, as well as the monitoring and output devices may be similar or identical. Likewise, the device can be used for surgery, procedural instrumentation, including catheters, and geoplasty devices, endoscopes, and the like. Likewise, quality control in food processing, and particularly in the meat and poultry industry, may find instant evaluation by nondestructive testing for tissue density, homogeneity, fat percent, protein content, possible disease or illness, and the like. Clean, quick, portable, onsite equipment may be available for testing of the subject tissue.

Similarly, in autopsies, determination of cause of death, extent of a terminal disease, extent of neurodegenerative diseases, and other unnatural causes, such as blunt trauma, poison, and the like may also be tested by characterizing them to create templates to which future evaluations may be compared. A device 210 in accordance with the invention may even be used to test artificially engineered tissues, biological materials, and the like in order to accurately characterize viable and biologically accurate microstructures.

It may test preservation of microstructures, test preservation of microstructural, or integrity of cryo-preserved tissues in organs for transplantation. Thus, the characterization of any tissue or material according to any criterion that can be identified and respond to ultrasonic pulses 208 may be characterized according thereto and evaluated in future instantiations.

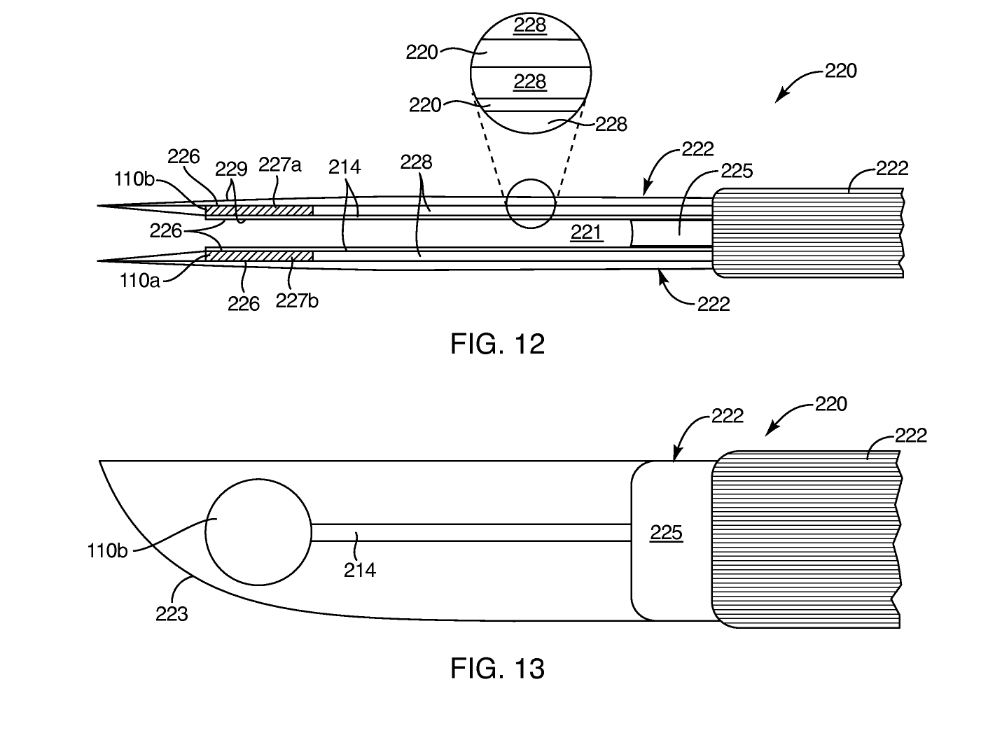

Referring to FIGS. 12 through 13, an apparatus 220 may be configured as a scalpel system 220 in which a handle 222 is secured to provide a surgeon with the ability to control a cutting edge 223 of a blade 224. In the illustrated embodiment, the blade 224 is actually two blades. Also, an armature 225 may withdraw entirely within the handle 222, when not in use, and extend out to the farthest extent of the blades 224. In this way, the armature 225 may clear the blades 224 of residual, tested material.

Meanwhile, electrodes 226 may be embedded within the blades 224, secured thereto, or the blades 224 themselves may actually operate as electrodes 226 in certain circumstances. Regardless, as in other embodiments, the piezoelectric elements 110a, 110b may be embedded in each of the blades 224. Accordingly, a certain amount of filler 228, such as a ceramic 228 may actually be used to shape the blades 224, thereby covering or otherwise protecting and hiding the electrodes 226.

In one presently contemplated embodiment, one or more electrodes 226 may be embodied in the metal of a blade 224. An opposing electrode 214 may be embedded within the filler 228 in order to access the opposing surface of a piezoelectric element 110.

In the illustrated embodiment, the opposing faces 227a, 227b of the piezoelectric elements 110 each require an electrode secured thereto. The electrodes 226 effectively operate at the outermost surfaces on the piezoelectric elements 110. In this way, voltage waveform may be applied to the piezoelectric elements 110 at their faces 227. This will cause a cyclical, piezoelectric distortion of the elements 110 as per the waveform. The electrode 214 serves as an electrode on the inside of a blade 224, while, in the illustrated embodiment, the blade 224, or rather metallic portion of the blade 224 may act as the opposite electrode 226 on the “back” of the piezoelectric element 110. In the illustrated embodiment, an insulating or other coating 229 may be provided on either or both sides or faces 227 of the piezoelectric element 110, or each of the blades 224 generally.

In operation, an apparatus 220 in accordance with the invention may include multiple electrodes 226 embedded within the filler 228. Alternatively, the electrodes 226 may be electro-deposited or otherwise attached as thin films to the outermost surfaces of the filler 228. By whatever mode, a pair of electrodes 226 may have a voltage applied at a specific waveform and frequency in order to generate the ultrasonic pulse through the appropriate piezoelectric element 110.

The gap 221 between the blades 224 will receive therein a specific sample 100, much as the forceps 180 of FIG. 7. In this instance, the blades 224 need not (but may) move together as would the forceps 180. The cutting edge 223 of each blade 224 will typically be centered along the thickness thereof. Accordingly, a certain wedging action will be imposed on tissue that is cut free by the blades 224, and imposed between the piezoelectric elements 110a, 110b. Thus, the scalpel 220 may operate with blades 224 at a fixed gap 221, which is therefore known. Accordingly, there may be no need to calculate a distance.

Nevertheless, the blades 224 may be instrumented as the forceps 180 in order to calculate, control, and otherwise know what the pressure is between the blades 224. Likewise, it may be beneficial to manipulate the blades 224 in some manner. In certain embodiments, the blades 224 may simply be fixed with respect to the handle 222 during use.

In the illustrated embodiment, the scalpel 220 forms a device for determining the pathology of biological tissue excised, or that continues to exist within a living body. Typically, in a situation that requires surgical incisions the dual-bladed scalpel 220 relies on the microscopic, ultrasonic, piezoelectric sensors 110 or micro-sensors 110 embedded in each blade 224.

One piezoelectric element 110 or sensor 110 in one blade 224 functions as the transmitter 112 while the other operates as a receiver 114 or a reflector 116. Small regions of tissue that are being cut by the blades 224 are probed by transmitting high-frequency ultrasonic waves, as described hereinabove, through the tissue captured between the blades 224. Because of the high-frequency, as described hereinabove, the transmitted ultrasonic waves provide time-domain and frequency-domain information.

That information is a function of the material properties of the microstructures of the cells and tissue in the sample 100. Thus, the pathology of the tissue may be assessed by the system 70. As described hereinabove, the transmitter sensor 110, 112 may be configured as a transmitter 112 and a receiver 114 combined to rely on a pulse-echo measurement as described hereinabove.

Again, small volumes on the order of less than 1 cubic millimeter of biological tissue in ex-vivo or in vivo environments may be assessed. Again, high-frequencies range typically from about 10 to about 100 megahertz and may be sensitive to a range of pathologies as described hereinabove. Likewise, piezoelectric elements 110 are comparatively thin, on the order of 50 microns for a 50 megahertz transducer producing optimal signals in the 10 to 100 megahertz range.

The insulating film 229 or coating 229 may be one of several types. For example, plasma-deposited ceramic layers such as aluminum oxide (Al₂O₃) or silicon dioxide (SiO₂) or a comparatively hard polymer film may serve as the layer. Likewise, other insulating films may be sputter-coated, plasma deposited, or otherwise applied.

Certain aspects provide unique benefits including the high-frequency ultrasonic transmitter 112 and receiver 114, which, again, may be embedded in a single piezoelectric unit 110. Again, in contrast to conventional medical ultrasound which uses diffuse reflection measurements, direct through-transmission and specular pulse-echo measurements may be taken directly. Likewise, the data analysis programs described hereinabove may determine the tissue pathology from the signals of the micro-sensors 110.

The application environments that do not have laboratories readily available in time or space due to their locations, the development of the country, and the like may be particularly well served by an apparatus 220 in accordance with the invention as discussed hereinabove with respect to other embodiments of the invention. Thus, guiding surgical and endoscopic procedures is available in real-time. Likewise, probing multiple tissue regions, or mapping specific locations within a region is available. Thus, the regions, the rate of change, and so forth may be determined by multiple tests at multiple locations within a region of a subject material 78.

Again, the applications to quality control for industrial processes, industrial materials, biological materials, meats, and so forth as described hereinabove also apply here. Likewise, as previous inventions, condition of meat in a restaurant, whether raw, partially cooked, thoroughly cooked, or otherwise prepared, may be evaluated in real-time. Sensors can be adjusted to compensate for temperatures. Likewise, quality control for measurement of consistency between batches in a product may be available by testing with an apparatus 220 in accordance with the invention.

The armature 225 may be automatically extended between the blades 224 periodically or after every test, in order to assure that each test is not affected by any residual material from a previous test. In another embodiment, a reverse-swipe arm or other method may clean the sample at each cut.

An apparatus 220 in accordance with the invention prevents loss of product because only a few cells need be sampled at a time. That is, the illustrations need not be contemplated as being exactly to scale in each dimension. Because only a few cells need be sampled at a time, homogeneity, protein content, percentage of various tissue types, and the like can be quantified by such information of the sensor system recognition program. This may operate similarly to methods used to model protein content and other pathological indicators such as cell size and density to identify human pathologies.

Because an apparatus 220 is configured as a scalpel 220 intended for medical use it can be incorporated into a surgical instrument 220 used for its conventional purposes. As the blades 224 are aligned closely enough to work as a unit this is entirely possible. In cases where a different density or protein structural quality has been reached, in any particularly delicate surgery is needed. A system 220 in accordance with the invention may be able to characterize samples 100 on-the-fly, in real time. For example, when a natural unfamiliar biological structure has been broken down due to infection or a cancerous or benign aberration within the normal tissue structure such anomalies may be detected. Also, their relative thickness can be determined.

For example, the shape of the blades 224, their spacing, and their relative thickness can be altered. By using condensation manufacturing techniques as described hereinabove, the blades 224 may be manufactured to be suitably sharp, precise, thin, and maneuverable. Likewise, they may be positioned to be touching, and an opening mechanism may be activated at any time during surgery for a sensitive ultrasonic measurement at a target spot.

For example, just as the forceps 180 open and close as a matter of utility, a set of blades 224 may open and close. This facilitates operating as a single blade 224 when closed together, and operating as a testing instrument sampling tissues when separated by a gap 221 therebetween.

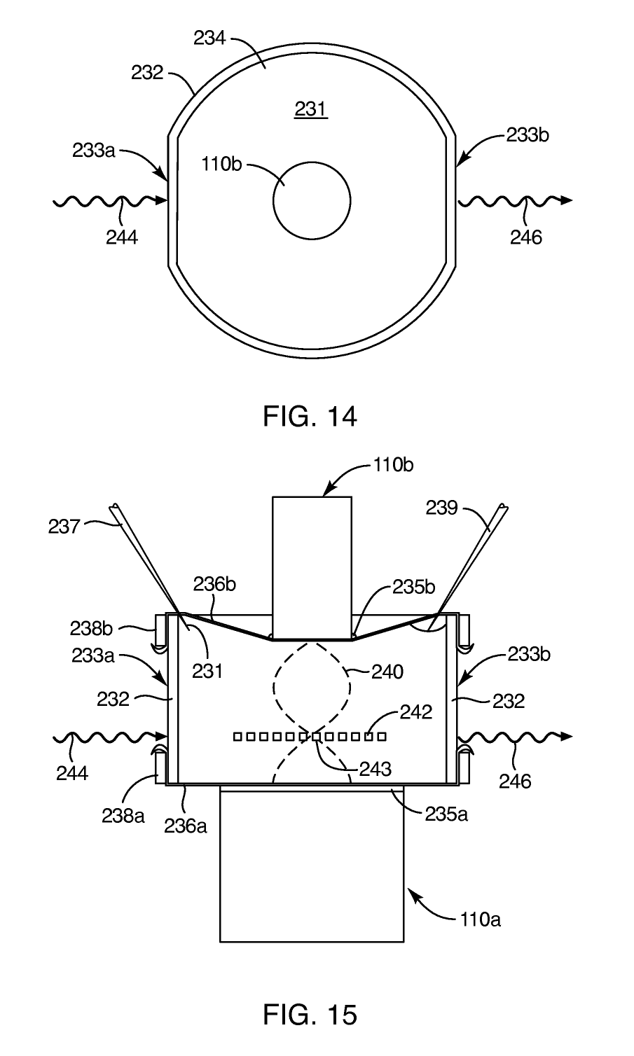

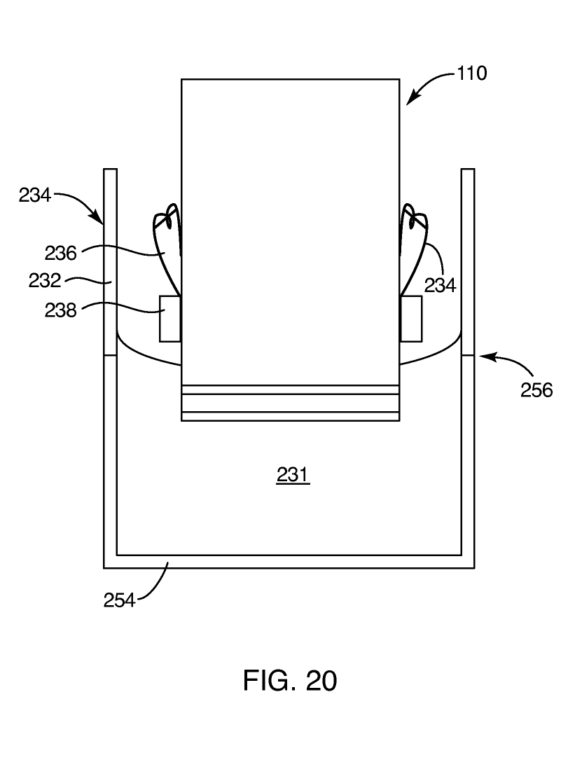

Referring to FIGS. 14 through 20, an alternative embodiment of a phenotyping system 230 may rely on a cell suspension 231. Alternatively, the material 231 may be any biological material or other material suitable for evaluation in accordance with the invention. Typically, a chamber wall 232 may define a region that presents two flat faces 233 on an otherwise cylindrical (by the mathematical definition) cavity 234 defined by the wall 232. In certain embodiments, notably FIGS. 14 through 18, as well as FIG. 20, a separator 236 may be typically embodied as a film 236 or sheet 236 of polymeric material. A polyethylene or other film 236 may provide a boundary to the cell suspension 231 within the chamber 234 or cavity 234.

In the illustrated embodiment, a needle 237 may be injected through a film layer 236b sealing a material 231, such as a cell suspension 231, within the cavity 234. Meanwhile, the film 236, may fit below as the floor or bottom film 236a, or above as the top film 236b. The films 236 may provide ends by being retained thereagainst by a retainer 238.

In the illustrated embodiment, the retainer 238 may be an elastomeric material that stretches to maintain the intimate contact and pressure against the film 236, thus securing it to the wall 232 of the chamber 234. In certain embodiments, a needle 239 may be used to remove bubbles or other materials at the top of the reservoir 231 or material 231. Likewise, the needle 237 may be used to withdraw a sample, such as may be used for chromatography, spectroscopy, or other analytical purposes. Likewise, a needle 237 may be used to inject chemicals, reagents, or other materials useful in preparing or testing a sample 231.

In the illustrated embodiments, a standing wave 240 may be established by a larger, low-frequency piezoelectric transducers 110. The standing wave 240 has the effect of suspending a cell layer 242 at a node of the standing wave 240. A separate, smaller, high-frequency piezoelectric transducer 110 may be configured to probe the suspended cell layer 242.

The layer of cells 242 may be impinged upon by light 244 passing through a transparent wall 232 and across the chamber 234. Accordingly, the light 246 passing out through the transparent wall 232 of the chamber 234 may be detected. Accordingly, the ultrasonic standing wave 240 may effectively set up the cell layer 242 for proper analysis according to the response of the cell layer 242 to the light 244 illuminating it.

In one presently contemplated device 230, the apparatus 230 may be set up to contain a cellular material 231 dissolved in some digest, such as a trypsin digest fluid and suspend it at the node 243 of the standing wave 240. In certain embodiments, one transducer 110 is used to create the standing wave 240 in a separate, high-frequency transducer is used to probe the cellular material 242 at the node 243.

In an alternative embodiment of the device 230, a combined dual-frequency, ultrasonic transducer 110 may create both the acoustic standing wave 240 and the high-frequency pulses that probe the cells 242.

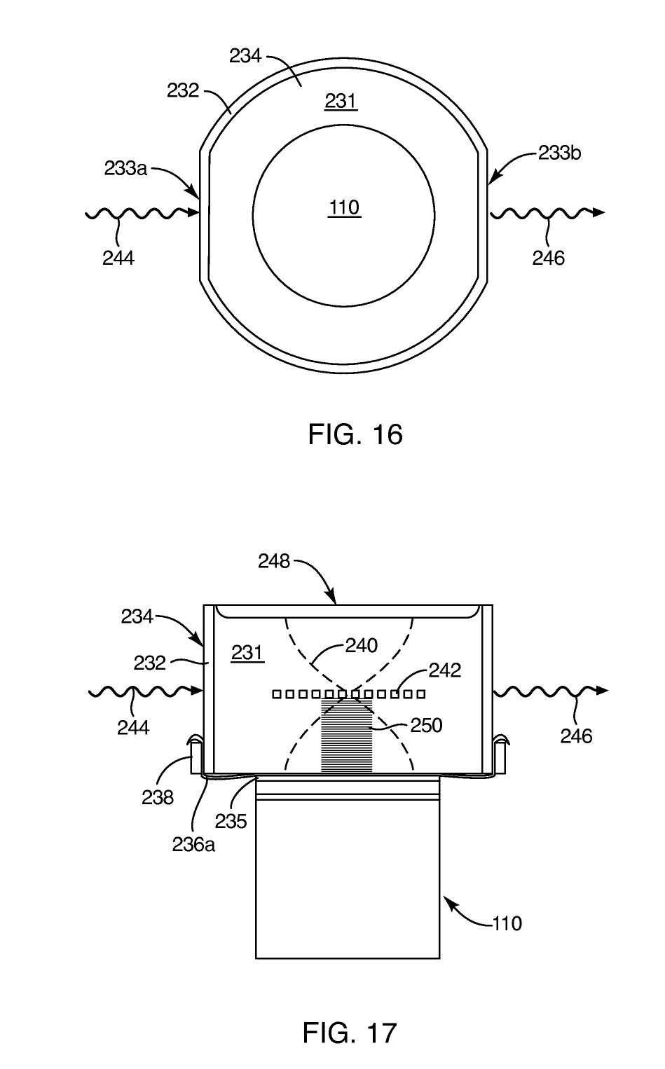

Referring to FIGS. 16 through 17, a single piezoelectric transducer 110 may be a combined dual-frequency ultrasonic transducer 110 capable of creating the standing wave 240 and of probing it as a transmitter 112 and receiver 114.

In the illustrated embodiments, a high-frequency transducer 110 transmits a high-frequency pulse, as defined hereinabove, which reflects off the cellular layer 242 at the node 243. The resulting waveform is then compared with a fluid blank. The fluid blank may be the material 231 that suspends the cells 242 therein. Such a sample may be taken before introducing the cells 242 that will eventually be driven to the cell layer 242. Thus, a frequency dependent representation (spectral analysis) or a time-and-frequency dependent representation (wavelet analysis) of the cell-dependent attenuation of the waveforms may thereby be provided from that comparison.

In certain embodiments, an optical sensor and probe may be positioned on the sides 233, or flat faces 233, of the chamber wall 232 allowing for quantitative optical analysis of cellular and extra-cellular features of the small sample cell layer 242. Such measurements may be taken in conjunction with the ultrasonic measurements taken by the transducer 110 or transducers 110. Such a chamber 234 is also known as a cuvette.

In the illustrated embodiment, the cylindrical chamber wall 232 contains the cell suspension 231 or tissue digest 231. As illustrated in FIGS. 14 through 15, a film 236b may seal the top of the chamber 234, just as the bottom film 236a seals the bottom thereof.

Referring to FIGS. 16 through 17, the top 248 may actually be open. Thus, when a pulse 250 at a high frequency is generated by the piezoelectric transducer 110, the reflection of that pulse 250 may be detected by the transducer 110 to acquire the data necessary for evaluating the cell layer 242.

A syringe or pipette needle 237 may introduce a cell suspension or other materials through the film 236b or directly into the material 231. Compared to the embodiment of FIGS. 14 and 15, wherein a low-frequency transducer 110a generates a standing wave, and a high-frequency transducer 110b is used for probing, the embodiments of FIGS. 16 through 20 illustrate a single transducer 110 that operates at both high and low frequencies to provide both the enforcement of a standing wave 240, and probing of the cell layer 242.

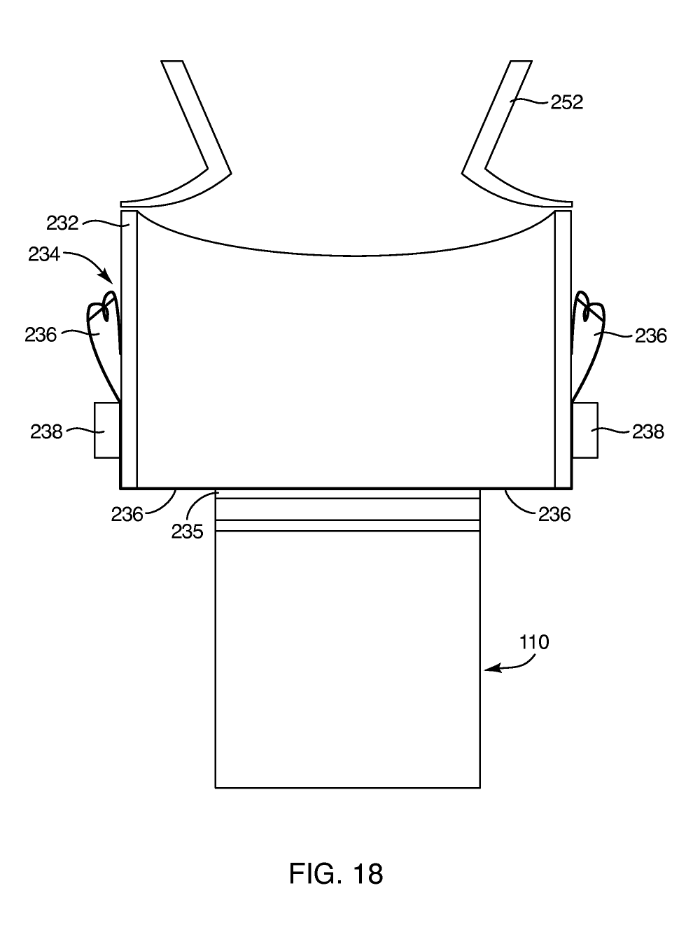

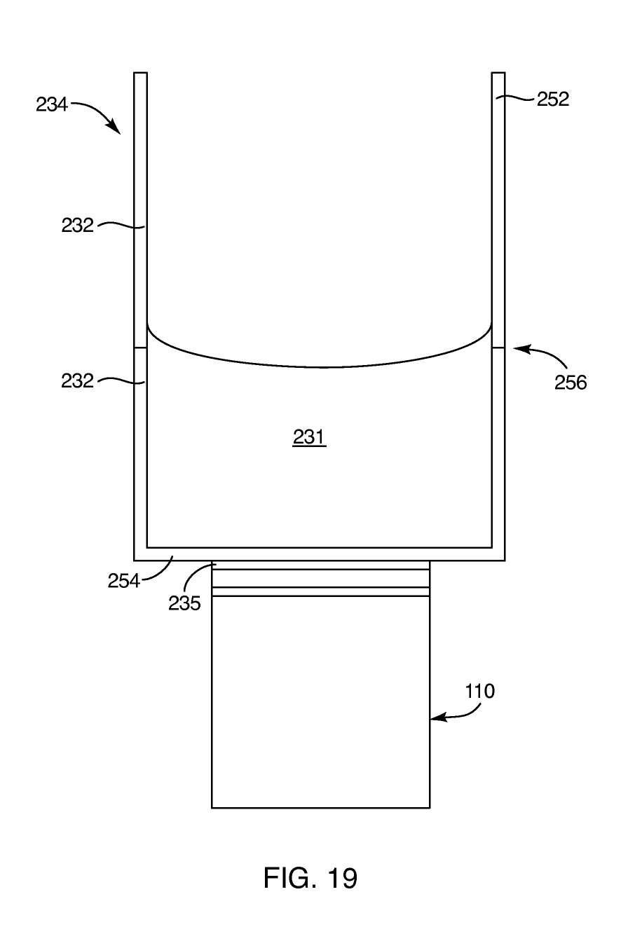

Referring to FIGS. 18 through 20, alternative embodiments may include, for example, a guide sleeve 252 to provide improved access, reduction of splashing or other disruption, or the like when adding cell samples to the bulk material 231 in the chamber. In the embodiment of FIG. 18, a film 236 is held by a retainer 238, forming the floor 236 of the chamber 234. In contrast, a rigid plastic floor 254 or base 254 in FIG. 19 acts as a transmission apparatus to transmit the waveforms from the transducer 110.

The entire system of FIG. 19 may be modified by inverting the transducer 110 to contact the material 231 in the chamber 234. As a matter of isolation, for protection, and so forth, as well as ready clean up, and the like, a film 236 may be secured by a retainer 238 on the transducer 110, rather than the wall 232 of the cavity 234 or chamber 234.

Referring to FIG. 20, in the illustrated embodiment, a drop guide may simply be built in as a comparatively higher wall 232, and a fill line 256 may be established for adding the material 231 to the chamber 234.

Typically, a cylindrically shaped cuvette 232 may contain a cell suspension 231 or tissue digest 231 and be open on both ends. Each of the ends may be covered with an optically transparent solid such as polystyrene, glass, or the like. Other plastics including polycarbonate and the like are also possible. Likewise, polyethylene or other polymeric films may serve. In the illustrated embodiment, the wall 232 itself in any of these embodiments may itself be optically transparent and comparatively rigid, stiff, and sufficiently strong to act as a container.

The polymer films 236 may then be used as covers in order to provide direct access or more direct access by ultrasonic waves to the suspension 231. The retainer 238 for holding the film 236 onto the wall 232 permits a single, closed end, or multiple closed ends. Thus, an air-fluid interface may be set at a specific height illustrated as a meniscus of the fluid 231 against the wall 232. The low-frequency element may provide the standing wave 240 and a high-frequency element 110 may provide the probing pulses 250 at the comparatively high-frequency, described hereinabove.

Typically, a cell layer 242 will actually be a mono layer 242 of biological cells 242 constrained by acoustic forces at the node 243 of the standing wave 240. In the illustrated embodiments, an apparatus 230 or cuvette system 230 may be tuned to a range of frequencies high enough to respond to cellular properties that would most distinguish between normal cells and those affected by disease.

For example, some diseases are accompanied or caused by a change in cytoskeletal protein content. This may affect the ultrasonic signal through a change in cellular bulk modulus. Short wavelength, high-frequency, ultrasonic pulses 250 are thus transmitted from a high-frequency transducer. That pulse 150 may thereby be reflected from the cell layer 242 and received back by the high-frequency transducer 110 to obtain a pulse-echo measurement. Signals may be amplified by receivers and high pass filters in order to separate the low-frequency standing wave 240, and its consequences, reflections, or harmonics from the high-frequency pulse-echo signal 250.

Signals may then be digitized, recorded, and converted to spectra. Spectra may be analyzed with principal component analysis (PCA) in conjunction with waveforms modeled on various sample properties in an analysis program connected with or incorporated into the system 70. Thus, the cuvette 230 becomes the probe 80 in the system 70.

The signal acquisition instrumentation may be connected to a mobile computer 126 to perform the analysis on site, and in real-time. Alternatively, analysis may be done after obtaining the waveforms. Spectra may be phenotyped with a map, correlating them to a specific molecular subtype or molecular pathology.

The amount of cellular material 242 may be measured by standard turbidity methods. Mie scattering may be used for an alternative confirmation of cell size. Also, principal component analysis may be used with the Mie scattering model to obtain cell size, refractive index, information about internal cellular structure, and so forth. Also, fluorescent biomarkers or Raman spectroscopy may be used to obtain more information on the chemical composition of the cells. Thus, the phenotyping capability may be enhanced to detect additional properties of the material 231, and specifically the cell layer 242.

Phenotyping cells by high-frequency ultrasound at frequencies sufficiently high to be sensitive to their biomechanical and morphological properties is a unique approach. Conventional isolation of DNA and testing for expression patterns is a mechanism for identifying nucleotides based on sequences. In contrast, apparatus 70, 230 in accordance with the invention combine both high-frequency ultrasound and acoustic separation in the suspension 231 of the cells 242.

This confines the cell layer 242 to a configuration that optimizes the high-frequency ultrasonic measurements. A planar, free-floating layer 242 of cells 242 is created in a cell suspension 231 or cell digest 231 by using a lower frequency, acoustic, standing wave 240. The cells 232 and other large cellular materials gather at the nodes 243 of the standing wave 240, thus separating them from other, typically smaller, dissolved components in the suspension 231 or medium 231, or digest 231.

The layer 242 provides a relatively flat reflective surface of cells, thereby increasing the amplitude of the pulse-echo signal 250 (see also pulses 106, 108 of FIG. 2). This improvement of signal to noise ratio in operation of the system 230 eases the analytical isolation of the waveform created by the cells 242 from those created by reflection off the walls 232 of the cuvette 230. Meanwhile, unwanted resonances of the low-frequency standing wave 240 may be separated out.

Meanwhile, phenotyping of cells by optical scattering and absorption is complimentary to the high-frequency ultrasound. Together they provide better and more information at better accuracy for use in diagnostics, qualitative analysis, qualitative analysis of cellular patterns, and the like within an individual or between and among individuals.

Thus, in summary, an apparatus and method in accordance with the invention may be used to analyze any cellular material for non-pathological effects that change cell size and phenotypic features. For example, many cancer types are first manifest by a change in the genetic expression profile of cytoskeletal-related proteins. Thus, properties such as cellular bulk modulus, nuclear size, and other properties detectable in the ultrasonic signal may be changed and detected.

Typical applications may include molecular subtyping of cancer cells, such as breast cancer cells, and in diagnosing diseases through samples collected from colon, prostate, or the like. Pathological conditions accompanied or caused by characteristic cytoskeletal dysfunction, such as neurodegenerative diseases, are also sensitive to the measurements made by such an apparatus 70, 230.

Inflammatory diseases from autoimmune responses, infections, and inheritance that result in breakdown in cellular, tissue, or both structures may be studied, diagnosed, or both by an apparatus 70, 230 in accordance herewith. Cell health may be monitored as in the case of chronic diseases such as diabetes with associated conditions such as neuropathy, vascular disease, kidney function, and so forth.

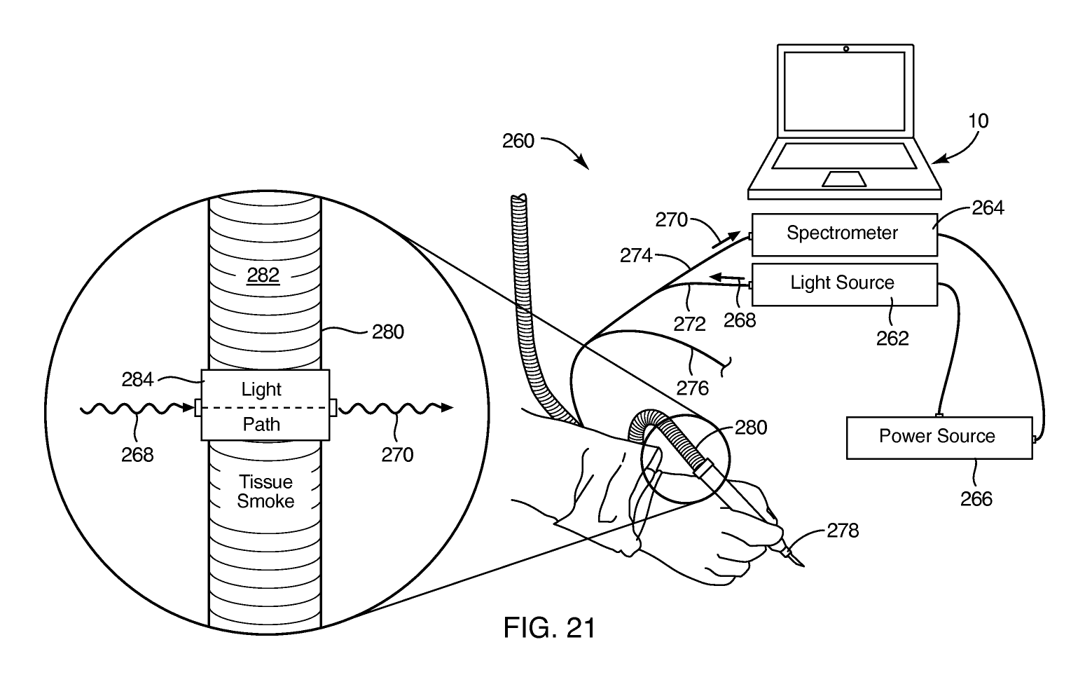

Referring to FIG. 21, in certain embodiments of apparatus and methods in accordance with the invention, a system 260 for aerosol spectroscopy may rely on a light source 262 in conjunction with a spectrometer 264. Both may be driven by a power source 266. Similarly, light 268 or light traveling in a direction 268 may flow from the light source 262 and illuminate a specimen of vapor, gas, or the like. The return light 270 or return direction 270 may be received by the spectrometer 264 in order to analyze the results of a surgery.

For example, a fiber optic cable 272 may have multiple fibers in order to pass light from the light source 262 toward a scalpel 278. The scalpel 278 is a self-cauterizing electrical scalpel 278 powered by a power line 276 or cord 276. The cord 276 may originate with the same power source 266, or may use another. In general, the power cord 276 delivers comparatively higher wattages as required for effectively evaporating tissues. In some embodiments, the scalpel 278 may be a laser cutter, a thermal cutter, or other electrical medical scalpel 278.

Typically, a conduit 280 must draw the smoke or vapors (burned and unburned molecules of tissue) from the site at which the scalpel 278 is working. Thus, the tissue aerosols 282 or smoke stream 282 from the electro-cautery or laser knife may be carried by the conduit 282 after being suctioned away from the incision site. The conduit 280 may pass through an observation box 284. The observation box 284 may provide a window through which light 268 passes through the aerosol 282 or smoke 282. The nature, material properties, and the like of the aerosol 282, smoke 282, or both 282 will influence the scattering, reflection, and so forth of the light 268 passed through the observation box 284 and resulting in the light output 270 transmitted back to the spectrometer 264.

The fiber optic cables 272, 274 carry the light 268, 270, respectively from the light source 262 and back to the spectrometer 264, respectively. In certain embodiments of an apparatus and method in accordance with the invention, such a system 260 may analyze or provide some reading that can be analyzed to identify tissues of cancerous composition versus healthy tissue. Various optical measurements such as Raman scattering, fluorescence, and spectroscopy in the visible through ultraviolet ranges may be applied to identify the constituents of the aerosols 282.

For example, distinguishing markers in the spectra may be identified by software comparing and contrasting samples from healthy and unhealthy tissues. Pattern recognition may be used as described hereinabove and in the references incorporated herein by reference. Learning programs may create acceptable databases 88 of information, templates, and the like.

In an apparatus and method in accordance with the invention, this optical method for determining the pathology of tissues based on cellular composition may identify cancerous cellular mutations that alter biomechanical and thus optical spectra associated with cellular material. Likewise, the evaporated counterparts will be chemically altered either in their chemical constitution or in their chemical proportions, and possibly both.

In certain embodiments, the altered optical spectra from variations in chemical composition provide to a surgeon the knowledge required to know when the electrical scalpel 278 (whether electro-cautery based or laser based) has transitioned from malignant cells or diseased cells to clean cells or healthy cells. Thus, a surgeon may know that a clean margin is being obtained. Likewise, an apparatus and method in accordance with the invention provides to the surgeon the knowledge of the various types of material, such as granular, muscular, connective, adipose, or other tissues.

In practice, optical signals may be obtained by through-transmission or by reflection. Thus, the “smoke” 282 or aerosol 282 retrieved through the conduit 280 from the cutting tip of the scalpel 278 may be sampled at a rate corresponding to the speed of cut, the speed of evaporation of the smoke 282 or aerosol 282, or both. This feedback provides to the surgeon real-time information regarding the nature of the tissue being excised.

Moreover, analysis may be dedicated, local, or online, in real-time. It can provide outputs by audio, visual, or both mechanisms. It may display on a monitor 122 an image, a picture, a graph, a chart, or other mechanism to inform immediately concerning the properties of the tissue subject to the scalpel 278.

In one implementation, the light source may be a laser or other lamp generating the light carried toward the scalpel 278 by means of the fiber optic cable 272. At or near the scalpel 278, including on the handle thereof, or along the conduit 280, light may be translated through the aerosol 282 being suctioned away from the incision site. By placing the fiber optic observation box 284 within the handle of the scalpel 278, or nearby, immediate feedback may be obtained.

Since the smoke 282 or aerosol 282 is considered a nuisance for smell, sight, and other effects, it needs to be suctioned away from the incision site. Thus, a second fiber optic cable 274 receiving the light 270 that has been transmitted through the detector box 284 or observation box 284 may be fed back into a spectrometer 264. The computer 10 connected to the spectrometer, the light source, or both may thus read the aerosol spectra from the spectrometer 264 and analyze the content of the aerosol 282 in a manner to detect the nature of the aerosol 282.

Pattern recognition programs, artificial intelligence engines, and the like as described hereinabove and in the references incorporated herein by reference illustrate mechanisms by which characterizations can be made with or without a full understanding of the structures. That is, one may recognize types by pattern recognition of spectra that characterize the microscopic structures of the aerosols 282. Those structures need not be quantitatively defined to know that their spectra identify them.

Gas chromatography may be linked to the scalpel 278 and used in rapid evaporative ionization mass spectrometry (REIMS) to distinguish between malignant and non malignant tissue. Testing by optical methods is capable of the speed and precision required to sample tissues at a much higher speed. Thus, outside of the laboratory, data gathered in accordance with the system 70, 260 in accordance with the invention may gather data in real-time as the smoke 282 or aerosol 282 is produced.

Accordingly, higher speeds are possible because optical measurements can be more sensitive to lower chemical concentrations, and a composition of the aerosol cloud 282 is sampled directly at the incision site with no separation process, delays in time, changes in temperature, and so forth. The production of sample spectra therefore need only be limited by the speed of processing by the computer 10. Time of flight, sensitivity, or aliquot size requirements associated with gas chromatography such as REIMS, need not limit the speed and precision of the system 70, 260.

The sampling speed will also more closely match the rate of incision speed and provide greater spatial resolution or more precise spatial resolution. Thus the potential for immediate feedback to give a clear picture of the sample area. It may do so in real-time to assist a physician in finding smaller extensions of cancer into healthy tissues. Such would often be missed by larger, more macroscopic assessments later in time, more distant in process, or the like. Likewise, such extensions may be missed by visual inspections. Thus, the optical path provides a greater average of the aerosol cloud making the measurements more uniform and statistically relevant.

In certain applications, a system and method in accordance with the invention may provide more precise excision of malignant and premalignant tissue during surgery with real-time intraoperative analysis of tissue. For example, obtaining cancer free margins in breast conservation surgery (lumpectomy), brain cancer surgery, Mohs surgery for melanoma, biopsies, exploratory procedures, laparoscopy, and the like may all benefit.

In the illustrated embodiment, the system 260 may use elastically or inelastically scattered light from the smoke 282, aerosol 282, being analyzed. Elastically scattered light (Mie scattering) may be analyzed with either reflection or extinction spectra. That is, reflection back or absorption in a through-transmitted or reflected illumination may be used. Likewise, inelastic scattering, such as Raman scattering may be analyzed using Raman spectroscopy techniques.

Meanwhile, fluorescence and absorption spectra may also be analyzed for additional or different information about the aerosol composition. Any of these signals or any combination of them may be gathered together to integrate the information regarding the aerosols 282 generated by the incision.

Studies have shown that aerosols can be differentiated based on their optical spectra. For example, principal component analysis (PCA) and other such methods have the capability of not only distinguishing between types of aerosols 282 by their refractive index, but also by other features such as particle size, size distribution, particle density, and so forth.

For example, the ultraviolet to visible extinction spectra have been used with PCA to identify aerosols of specific sizes and size distribution. These two properties may vary between normal and malignant cells in tissue smoke 282 or aerosols 282. Due to different droplet sizes produced by compositional variations in the evaporated lipid compounds, such characterizations are readily possible.

Various aerosol sizes and size distributions have demonstrated an effect on Mie scattering and can be distinguished by extinction spectra through PCA, other pattern recognition programs, artificial intelligence, comparisons, and the like. Comparison of simplified systems of cellular components and theoretical modeling of spectral changes may also provide means to identify what compounds are causing changes in spectra. Thus, accumulation of a database 88 of such testing permits investigation of several methods for analyzing tissue smoke aerosols 282.

Bovine tissues may be used as models in establishing a database 88 and various tissue smoke 282 may be compared in order to determine light frequencies (colors), the spectra for use as the incoming light 268, likely shifts, various scattering spectra, and so forth. Optical sensors may also be evaluated for minimum and maximum sensitivity to various tissue types, density of smoke, and so forth. For example, certain frequencies of light may pass through smoke more readily, therefore certain particle types and sizes may be more readily detected.

Particle sizing may be considered also and analyzed through other methods. Thus, determining how particle sizing affects the precision and the characterization may be studied by such a method. Comparative mechanisms such as comparing healthy versus cancerous tissues and building up the database 88 will be able to improve the distinguishing more readily between various tissue types. Thus, the fine line between cancerous, pre-cancerous, and non-cancerous tissues may be thereby distinguished in order to assist in determining the margins of tumors.

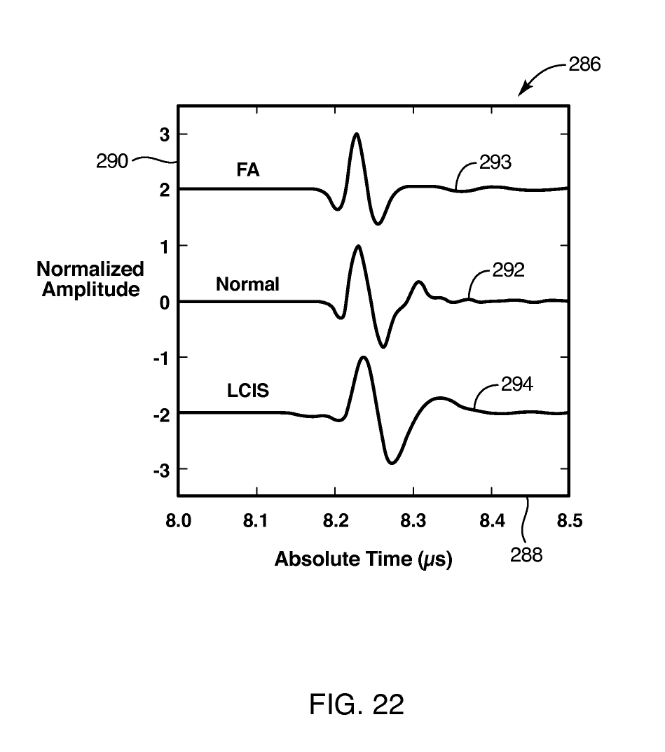

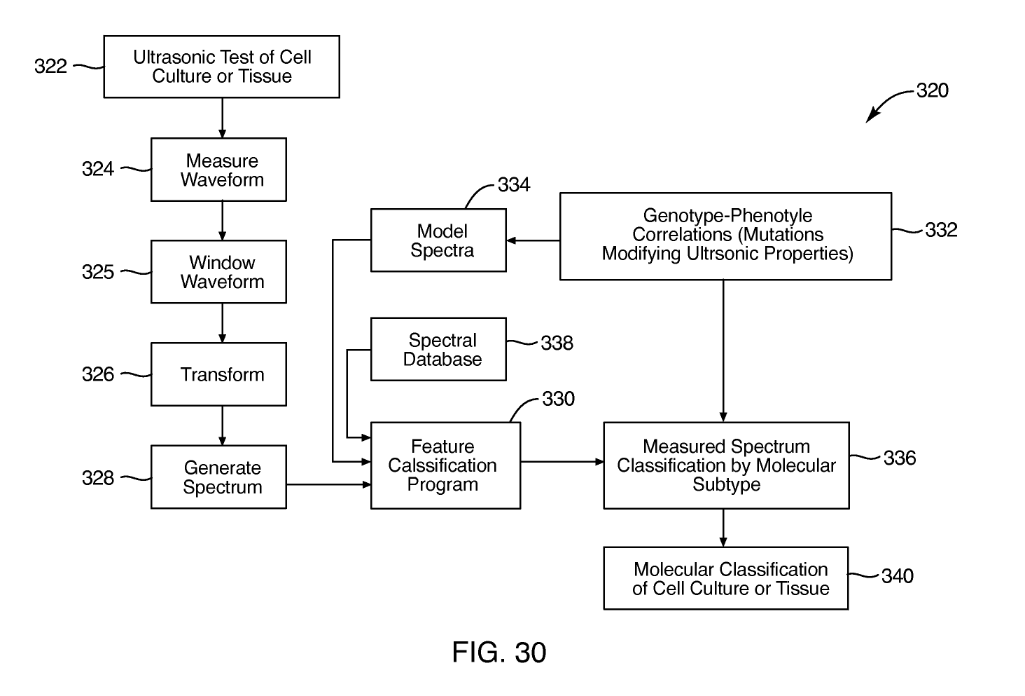

Referring to FIGS. 22 through 29, certain methods and apparatus are effective for determining tissue type (microstructure or histopathology), while referring to FIGS. 30 through 37, certain methods and apparatus are effective for determining molecular subtypes. These may come from various cell cultures, excised tissue specimens, in-vivo measurements, and the like. They extend to industrial materials, industrial processes, and so forth. Measuring an ultrasonic waveform in a material of interest may characterize such materials at a much more resolved (e.g., more detailed, more specificity, smaller scale) than even the tissue typing discussed hereinabove. Ultrasonic waveforms may be acquired at frequencies that are selected to be particularly sensitive to various properties of cells.

For example, cellular phenotypes, may be characterized, and are affected by the morphology (e.g., size, shape, components, structures) of a particular cell as well as the biomechanical (mechanical properties such as bulk modulus, density, shear modulus, and so forth) of individual cells, and individual cell components. Thus, one may subject a cell culture to ultrasonic waves and analyze the through-pulse or reflected-pulse traces thereof in order to characterize cells by molecular subtypes or cellular subtypes.

As discussed hereinabove, waveforms used to shape the ultrasonic pulses may be received as through pulses or as reflected pulses. The altered waveform received may be analyzed to provide either frequency-dependent representations (spectral analysis) or time-and-frequency dependent representations (wavelet analysis) of those returned, altered waveforms.

Likewise, model-based representations may also be used to classify the physical data collection according to phenotype using a classification algorithm described hereinabove. Thus, at a more detailed level than tissue typing, an apparatus and method in accordance with the invention may be used for molecular subtyping or genotyping determined by their correspondence to phenotyping.

In certain embodiments of an apparatus and method in accordance with the invention, one may determine molecular subtypes of various cells, cell cultures, or the like. This may be done in-situ or ex-vivo. As a practical matter, the method relies on the scientific observation that different molecular subtypes of a particular cell type, or malignant cell type will exhibit various mutations. Each may be associated with actin cytoskeleton, the extracellular matrix (ECM), integrin signaling systems, and so forth.

However, the mutations that affect those characteristics of a cell also alter mechanical (think mechanical properties) characteristics of the cells as well. Thus, in order to identify certain molecular subtypes or cellular subtypes, one need not go to the genetic or chemical level, or any other similarly detailed features in order to determine a classification. Instead, in accordance with the invention, one may use ultrasonic probing to characterize the biomechanical properties that are sensitive to ultrasound, thereby identifying subtypes correlated thereto. In this way, it has been found that one may characterize genotype by identifying ultrasonically determined phenotypes based on mechanical properties.

As a practical matter, high frequencies in ultrasonic pulses may be transmitted through cell cultures, tissue specimens, regions of the body, industrial materials, industrial processes, foods, and the like and received by sensors positioned for through-transmission or pulse-echo recording. The time-domain waveforms are configured as frequency-domain spectra using Fourier transforms as discussed hereinabove.

Meanwhile, the pulse power spectra may be analyzed by comparing them to previous measured spectra, to modeled spectra generated by numerical methods simulations (a defined mathematical and computer term of art), or both. As discussed hereinabove, one may use such modeling to characterize or classify empirical data. One may also use previously taken databases of empirical data to characterize current empirical data. Likewise, one may use empirical data in order to refine the accuracy of a model-based system.

In addition to Fourier transforms, other methods of transform exist also, and are equally applicable. For example, wavelets may be thought of as a three dimensional surface in a space characterized by one axis representing time, another axis representing frequency, and a third axis representing an amplitude of a waveform signal. As a practical matter, such can be imaged or represented on a display with color, or with amplitudes in a three-dimensional image representing the surface.

Thus, various transforms have been used in an apparatus and method in accordance with the invention. Each may be used to characterize the waveform responses detected by sensors. Those transformed spectra resulting may be analyzed according to methods in accordance with the invention.

Referring to FIG. 22, a chart 286 is defined by an x axis 288 or domain axis 288, and a range axis 290 or y axis 290. In the illustrated chart 286, a curve 292 represents the normalized amplitude of a voltage representing the ultrasonic waveform received through a normal control specimen from a through-pulse transmission. The other curves 293, 294 represent fibroadenoma (FA) and lobular carcinoma in situ (LCIS), respectively. Each of these curves 293, 294 has been offset by a value of positive two and negative two, respectively, along the y axis 290 for clarity in distinguishing the curves 292, 293, 294. One will notice that each of the curves 292, 293, 294 has somewhat different, yet somewhat subtle, individual artifacts.

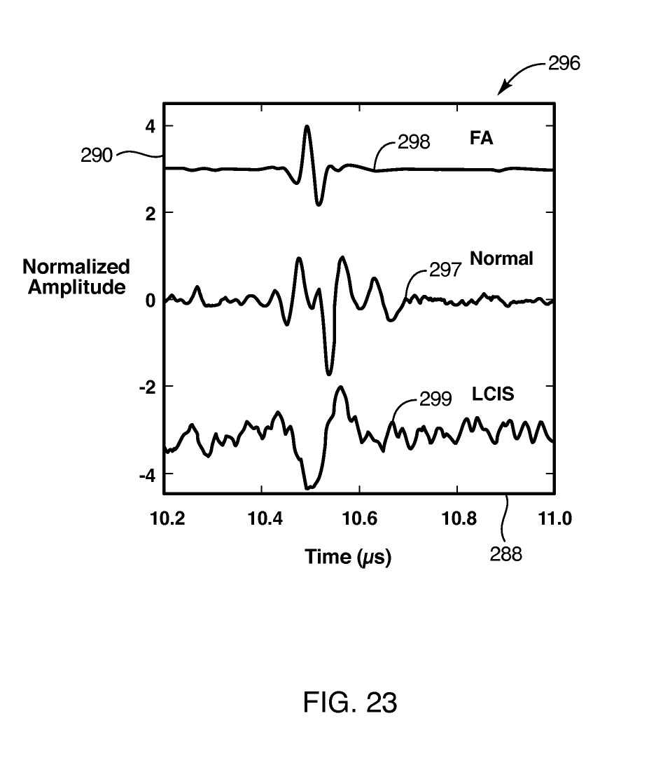

Referring to FIG. 23, a chart 296 illustrates the normalized amplitude of voltage from a sensor detecting an ultrasonic pulse of a pulse-echo configuration in a time domain. The curve 297 represents a normal cell type, whereas the curve 298 represents a fibroadenoma and a curve 299 represents lobular carcinoma in situ malignancy. Again, the curves 298, 299 have been offset a distance of positive 2.5 and negative 2.5, respectively, along the y axis 290 from the normal curve 297, for purposes of clarity.

Again, one will note various artifacts in each of the curves 297, 298, 299. In particular, one will note that not only are the curves 297, 298, 299 characterized by more peaks and valleys, but the tissue types differ as well, somewhat more dramatically. However, a certain amount of the variation in each of the curves 297, 298, 299 is characteristic of various reflections better characterized as noise.

Some of this noise is due to the fact that the waveform of the ultrasonic pulse 106 is being propagated through a specimen, then is bounced from a reflecting platen or anvil. Then, the waveform comes back through the specimen again. Moreover, the transducers 110, themselves likewise have multiple echoes within each transducer itself, and so forth.

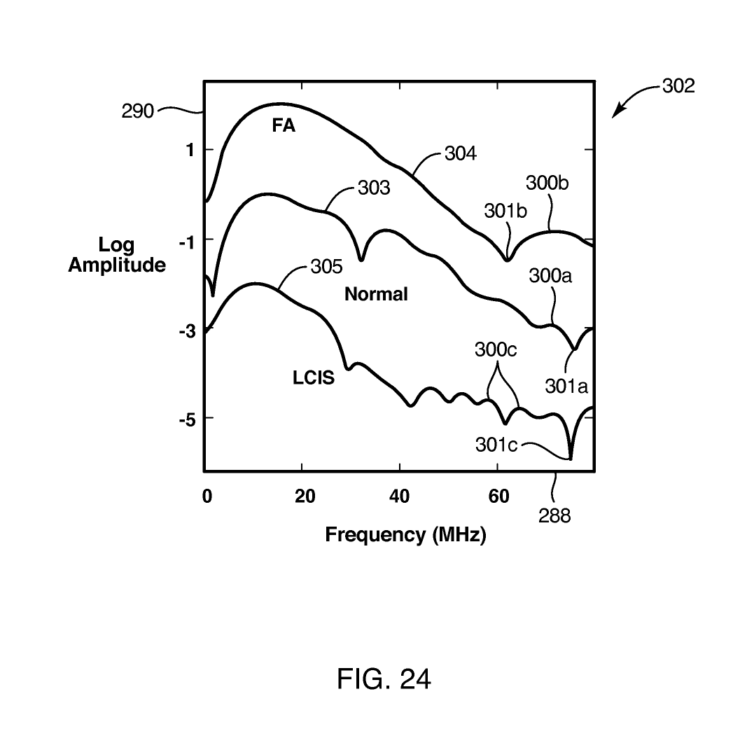

Referring to FIG. 24, a chart 302 plots a log of amplitude, representing a power spectrum in a frequency domain, represented along the x axis 288. These curves 303, 304, 305 correspond to the data of curves 292, 293, 294 of FIG. 22. That is, the data in the chart 302 is based on the same tests, same tissue types (microstructure or histopathology) of tissue samples, waveforms, and sensors as the chart 286. However, the chart 302 is used to characterize the various curves 303, 304, 305 according to their peak densities.

For example, one will note that each of the curves 303, 304, 305, representing its particular tissue type (microstructure or histopathology) as discussed hereinabove, may have various peaks 300 and valleys 301. For example, the peak 300a corresponding to the normal curve 303 is distinct, in its place along the frequency domain 288 compared to the peaks 300c and 300b of the respective curves 305, 304. Likewise, the number and positions of peaks 300 and valleys 301 for each of the curves 303, 304, 305 may vary. As discussed hereinabove, one may phenotype each of the samples according to a peak density analysis described hereinabove.

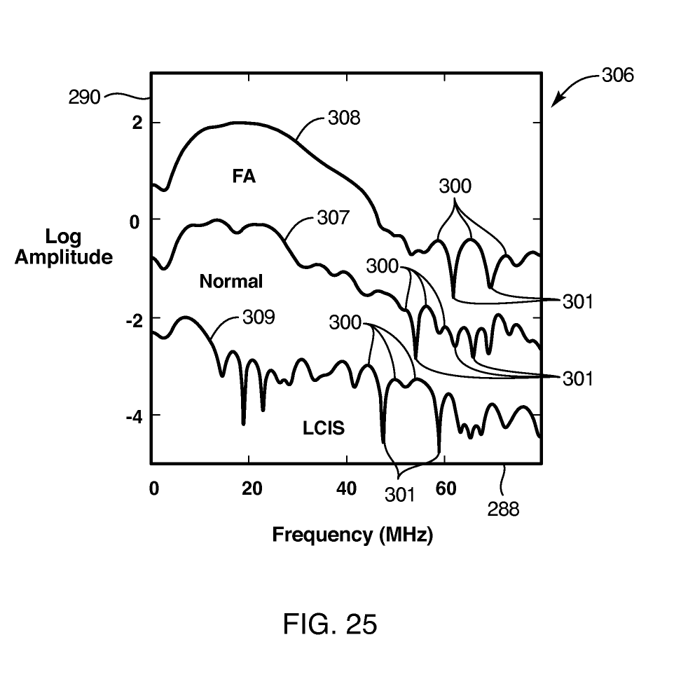

Referring to FIG. 25, a chart 306 represents three curves 307, 308, 309 again having various peaks 300 and valleys 301 from the pulse-echo experiment of FIG. 23. One will note that without resorting to the time domain in FIG. 23, one may immediately characterize each of the curves 307, 308, 309 according to the peak densities. The analysis is even more telling when considering various bands within the frequency domain 288 thereof. For example, between 0 and 40 megahertz, the curve 308 has very few peaks, the curve 307 has a few more, and the curve 309 has several. Meanwhile, in the range of 40 to 80 megahertz, each of the curves has various peaks 300 and valleys 301, and at characteristic positions and numbers.

Thus, as discussed hereinabove, in a method and apparatus in accordance with the invention, one may characterize tissues and cells by phenotype based on the ability of the mechanical properties and microstructures of the tissues to scatter either through-pulses or reflective-pulses of ultrasonic waves.

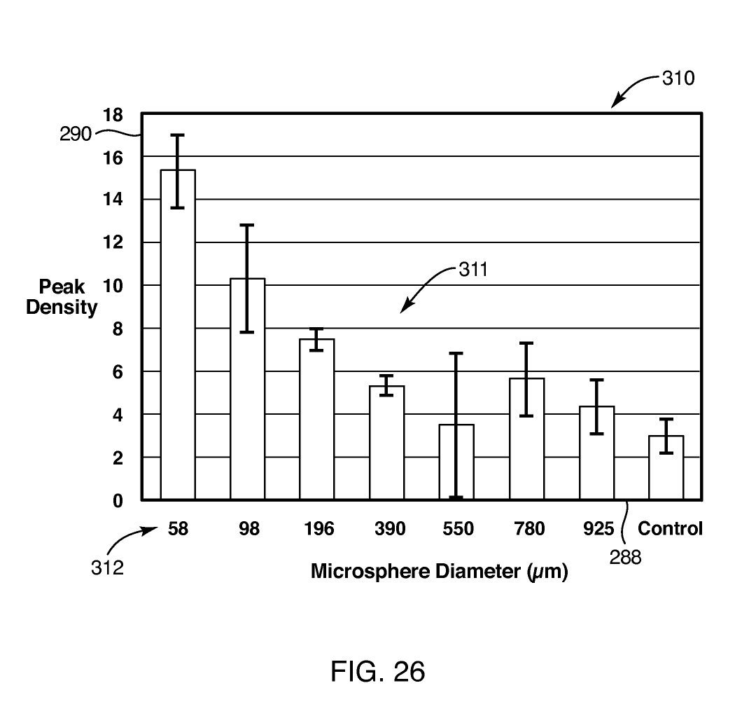

Referring to FIG. 26, in contrast to the actual breast tissue specimens used in the experiments of FIGS. 22 through 25, a chart 310 represents values 311 of peak density corresponding to a completely inanimate material. For example, in the illustrated chart 310, high frequency ultrasonic data was collected for various micro-spheres, represented by diameters 312 along the x axis 288. Accordingly, along the y axis 290, values 311 of peak density were recorded for each. One will notice that a clear monotonically decreasing curve may be fit through the center of the error bars of the first five samples.

Perhaps due to resonance or some other characteristic of the experiment setup, a rise occurs in the values 311 for the next sample, but again monotonically decreases through the remainder of the samples. A control is also represented and so identified.

The peak density values 311 clearly demonstrate the trends with micro-sphere diameters in the range of 58 to 925 microns. The peak density analysis is quite robust in its ability to differentiate between micro-structures of particular materials of arbitrary configuration, material, and disposition within a matrix.

Thus, it can be seen that materials in industrial processes, such as food processing, emulsions, mixes, and so forth for industrial materials, industrial processes, food processing, quality control, process control, and the like may be tested, controlled, and otherwise characterized by methods and apparatus in accordance with the invention. Moreover, various other particulates disposed within matrices of different materials, from rocket fuels, to aerospace materials, particular composites, filled structural materials, concretes, and the like may respond to tests in accordance with the invention.

Classically, fiber composites have been used in order to improve the overall properties of structural materials. Such materials may be characterized with through-body (entire part) or by sampled layers, by destructively obtained samples, or by non-destructive testing in accordance with the invention. Scanning electron micro-graphs are often taken by electron microscope to characterize granular samples of alloys, and so forth. Alloys derive substantial variation in control of material properties such as stiffness, strength, and so forth from the microstructure of granular developments within the alloy. Such micro-structures may likewise be characterized by apparatus and method in accordance with the invention.

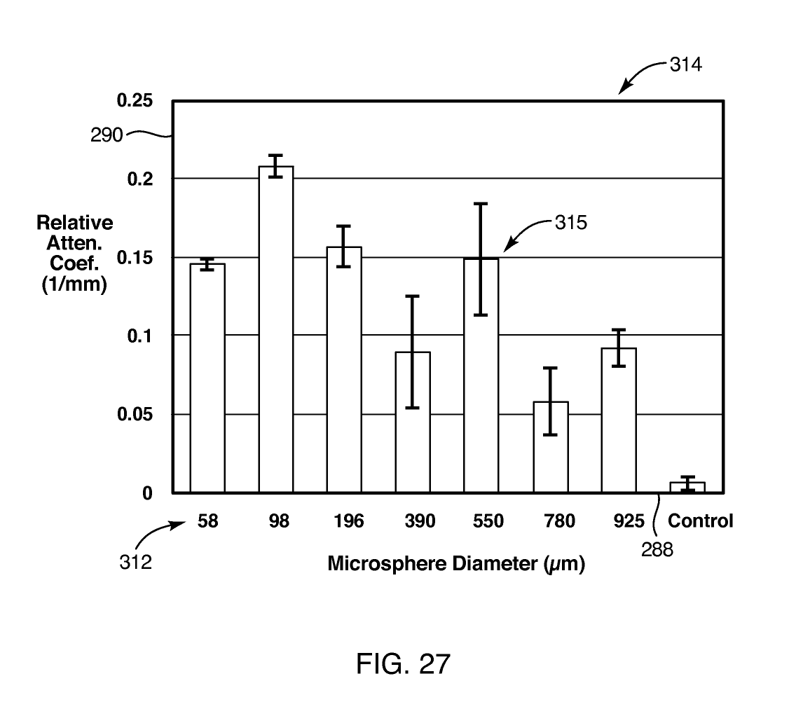

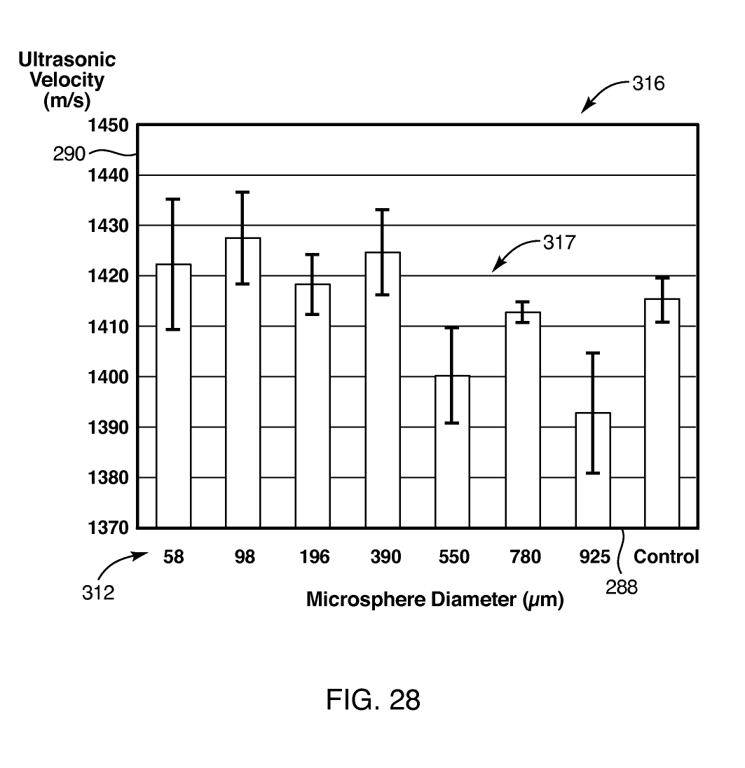

Referring to FIGS. 27 through 28, one may notice that the trends identifiable in the chart 310 are not as consistent nor evident in the charts 314, 316. For example, chart 314 represents relative attenuation coefficients in terms of inverse millimeters along the y axis range corresponding to the range of diameters 312 along the x axis 288. One will notice that a pattern is not distinguishable in those first five data sets. Likewise, no trend is identifiable in the following three data sets.

By the same token, no more resolution is identifiable in the chart 316. The values 315, 316, 317 do not correlate by any obvious mechanism to the values in the respective domains 312. As a practical matter, it is valuable to realize that peak density analysis is not classical attenuation theory or attenuation measurement. Similarly, what should be clear in the process described hereinabove is that neither attenuation nor velocity variation is the same as peak density analysis. Moreover, the results thereof do not correlate.

Referring to FIGS. 26 through 28, the high frequency ultrasonic data of peak density was developed by creating a tissue analog made up of gelatin and soluble fiber. Embedded within that matrix was a distribution of polyethylene micro-spheres. In each specimen, a constant volume percentage was maintained at 0.8 percent. Again, the micro-sphere diameters range from 58 to about 550 microns.

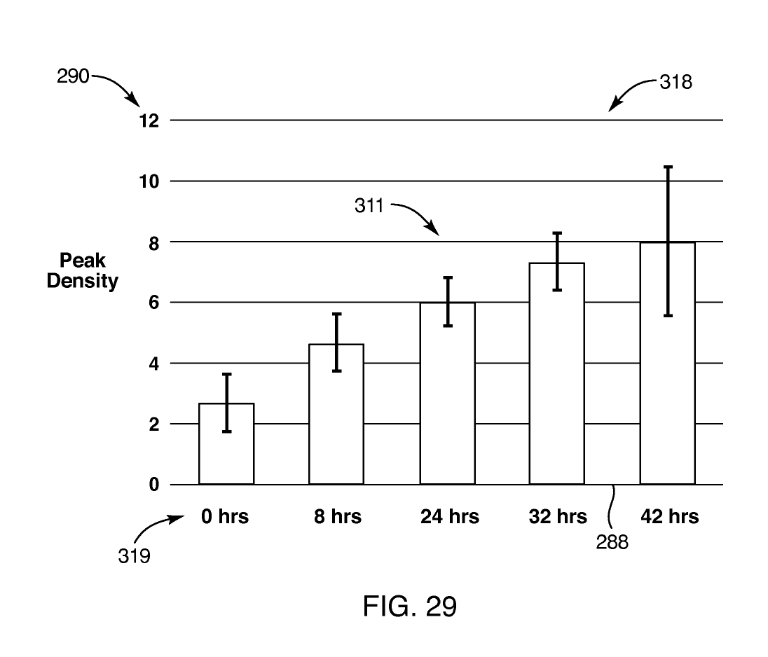

Referring to FIG. 29, the chart 318 represents various values 311 of peak density corresponding to various times distributed along the x axis 288. In this experiment, high frequency ultrasonic data from tissue specimens were taken. The specimens were excised porcine heart muscle undergoing fixation in formalin.

The peak densities 311 illustrate that the formalin is preferentially changing the properties of certain components within a cellular structure. It is clear that the uptake rates are changing the properties of certain components more compared to others. The microstructure is being altered by the fixation in formalin. There becomes an increased spatial heterogeneity of the microstructural properties. Thus, there becomes an increased level of ultrasonic scattering and the resulting values 311 of peak density 311 as distributed along the y axis 290.

Referring to FIG. 30, an apparatus and method in accordance with the invention may be applied to individual cells. For example, a method for determining molecular subtypes of cell cultures, industrial specimens of materials, tissue digest, food products, end products or immediate products in industrial processes involving particulates, and the like may be characterized.

This is based the fact that ultrasonic waveforms may be acquired at frequencies that are sensitive to the microstructures of such particulates. These are due to the morphology (think in terms of size, shape, and other geometric variables) and biomechanical properties (think in terms of bulk modulus, shear modulus, and other characterizations of stiffness, density, attenuation, reflection, elasticity, inelasticity, and so forth). In a method in accordance with FIG. 30, a process 320 may begin with testing 322 a cell culture. Again, cells are used by way of example, because they provide a particularly useful application of an apparatus and method in accordance with the invention. However, what is stated here for cells applies generically to the industrial processes, materials, and so forth mentioned hereinabove.

Testing 322 of a cell culture may involve identifying a sample, preparing a specimen, and subjecting that specimen to an ultrasonic pulsing by any of the mechanisms and devices described hereinabove. Thus, testing involves preparation of a sample, subjecting it to an ultrasonic pulse regimen, in one of the devices 80 in accordance with the invention, in applying an ultrasonic pulse.

Measuring 324 the waveform represents the process of acquiring the signal of the applied wave 106, after having passed through the specimen. It has been found for cell cultures that a pulse-echo has been found entirely satisfactory. This is especially true for a monolayer cell culture. Cultures are often made on a flat substrate of a growing medium. They tend to propagate in comparatively thin, often monolayered, cultures. Thus, a single cell type (mono culture) may be grown in a monolayer configuration due to its growth environment. Thus, it has been found not only suitable, but quite satisfactory to use reflected wave or pulse-echo methods due to the reduction in noise, echoes, interference between waves, and between fixtures and other structures.

As a practical matter, the possibility of an acoustic impedance mismatch due to a comparatively thin cellular layer and its proximity to its supporting substrate, such as a well floor, or the like introduces clutter (noise) in the retrieved signal. Thus, a reflected pulse 108 operates better than a through-pulse 106, contrary to the preference in a bulk culture such as described hereinabove.

Windowing 325 a waveform may be thought of as isolating the particular response waveform 108 that is received back by the receiving transducer 114. One desires to analyze the portion of the overall response that represents the time of interest. That time of interest will contain the artifact represented by the influence of the cell culture on the waveform. Thus, windowing 324 or isolating the time segment or time trace of the received waveform may often be done by recognizing the characteristic shape of the waveform, and thereby selecting a beginning time and an ending time to be considered.

As discussed, with respect to various hardware and methods, transforming 326 may be done by any one of several available methods. For example, a Fourier transform may be applied to the waveform windowed 325 and received from the testing 322.

Transforming 326 does not require the double transformation discussed above. Rather, here a single Fourier transform applied to the waveform, may then have its absolute value taken to generate a curve that represents the power spectrum. Measuring 328 that spectrum involves generating the power spectrum from time domain data transformed.

Generating 328 a power spectrum results from taking an absolute value of the output of the transformation 326. The absolute value of a Fourier transform 326 of the waveform is the power spectrum. With the power spectrum, it has been found that the graph or curve of that power spectrum differs from the method using the second transform, and taking the slope thereof described hereinabove. However, it also differs from the peak density method described hereinabove, which also relies on the absolute value of a single Fourier transform to obtain a power spectrum.

Because a process 320 is typically characterizing a single cell type, and often in a very thin or even single-cell layer, the curves 292, 293, 294 and especially the curves 297, 298, 299 are simpler. Thus, whereas curves 302 and 306 were calculated to simplify the almost insurmountable complexity of the curves 292, 293, 294, 297, 298, 299, the curves 307, 308, 309, representing the pulse-echo response are greatly simplified.

Accordingly, a feature classification 330 is possible without resorting to peak density analysis. Feature analysis 330 applied to the data of the chart 306 is complex, and not clearly correlative. In contrast, applying the generation 328 of the power spectra to thin layers or single layers of cells, feature classification 330 becomes quite tractable (for example, see the remaining charts hereinafter).

Feature classification 330 may actually rely on other data that may exist in correlations 332 of other genotypes or phenotypes that have been achieved by conventional or classical methods. For example, various correlations may be obtained from characterizing specific cell cultures that are well established and available for characterizing malignancies, normalities, and the like. These may be databased after extensive research, tremendously complex data analyses, and the like. Accordingly, a database 332 or correlations 332 may actually be used in feature classification 330.

Likewise, modeling 334, as has been described hereinabove, may be relied upon to model the power spectra for feature classification 330. For example, the modeling procedures described hereinabove identify the characterizing properties and morphologies (size, shape, etc.) of cellular and tissue microstructures, in order to model their mechanical responses to ultrasonic pulses. Such modeling 334 may be used as an input to feature classification 330. Empirical correlations 332 or databases 332 thereof as well as modeling 334 may be used in feature classification 330. Moreover, the actual databased correlations 332 may also be used in modeling 334, either as testing, proof, or for calibrating and adjusting for accuracy the analytical models relied upon.

Correlating 332 genotypes, phenotypes, and so forth to various cell types, tissue types, or the like may be done by a variety of classical techniques. These typically take extensive equipment, considerable time, and substantial data processing. As a practical matter, there is no need to ignore the value of such data. Thus, correlating 332 may result in databasing 332 correlations.

Thus, in modeling 334 the power spectra of various cell types, the correlations 332 may be readily available for certain cell types based on classical analysis, microstructure analysis, and actual physical measurements of properties thereof. Modeling 334 may rely on those correlations 332 for the physical properties and microstructures with which to analytically model tissues, cells, industrial fluids, solids, gels, particulate materials, and the like.

Meanwhile, spectra databases 338 may represent empirical data gathered from types of cells that have been characterized or otherwise known, and which have been subjected to ultrasonic pulsing, in order to characterize them. The informed modeling 334, as well as the empirical spectral databases 338 may be used in addition to, instead of, or in any subcombination, with the generated 328 spectra.

Feature classification then involves a process of characterizing the curves of data representing power spectra corresponding to cell cultures across some frequency domain. For example, various artificial intelligence engines are fully capable of classifying or characterizing relationships between curves. For example, described hereinabove, are the Cook references that rely on very little understanding of the sources of differences, but simply characterize, correlate, and classify differences.

Likewise, methods such as principal component analysis are well established and described in the art for characterizing curves. Moreover, other more rudimentary mathematical and engineering analyses, such as peak height, maximum width at half peak height, relationships between centroids of area, characterizing symmetry or asymmetry, and so forth may also be used from classical mathematics and numerical methods to characterize various curves. As a practical matter, in ultrasonic measurements, peak heights are not particularly useful. However, the actual location along the frequency domain at which the peak is found may often correlate and distinguish particular cell types.

Thus, feature classification 330 is a matter of choice. However, choice is guided by those methods of classification and characterization that seem to provide reliable, repeatable results for classifying a particular cell culture.

As a practical matter, classifying 336 by molecular subtype the particular spectrum being analyzed, may have various implications. For example, in medical or biological contexts, one often knows already the type of cancer, and is more interested in distinguishing a subtype that will characterize a suitable treatment.

For example, certain subtypes of particular cancers respond to chemotherapy, others to radiation, others to hormonal therapy, and so forth. Thus, a particular type of cancer will not necessarily respond to all types of treatment. Instead, different subtypes will respond to different, specific treatments. Thus, the subtype is very important. However, subtype may be a characterization in a type that is already known.

In contrast, classifying 336 the subtype followed by classifying the type or general classification is more important, perhaps, in an industrial context. Where one does not know a type, the subtype may so indicate. Thus, a particulate contaminant, a grain structure in a micro-graph, a pharmaceutical sample, a binder in a particulate material, a contaminant or constituent out of specification within a composite of particles and excipients in a pharmaceutical preparation, or the like might not be pre-characterized by type. Accordingly, the subtype classified 336 may then enable classification by type.

For example, in drug preparations, a drug may be compounded as a mixture of comminuted (powdered) constituents mixed in a certain ratio. These may be enclosed within a gelatin or other capsule. In contrast, such constituents may be mixed in a similar manner or identical manner, and then compounded with other excipients, such as binders, flow agents, and the like in order to form a pill. Quality control may be effected by a process 320 in accordance with the invention by analyzing such products, either as the bulk mixture of constituents, or the compounded pill, capsule, or the like by the process 320. Accordingly, verifying that the proper amount of the proper constituents and all the proper ratios exist may be evaluated. Similarly, contaminants, percentages and integrity of binders and the like may also be evaluated.

In another example, separation of emulsions may be detected. For example, an emulsion is well characterized by vinegar-and-oil salad dressing. In such an emulsion, one liquid is dispersed within another liquid. Separation is a function of time. In testing emulsions, one may characterize those emulsions very rapidly in a dynamic setting due to the speed at which the parsing (separation and identification) may be done in accordance with the invention.

Thus, quality control, process control, verification or testing of the time of separation, the rate of separation, and the like may be done. By conventional methods, considerable equipment, expense, and time may be required. However, in a method 320 in accordance with the invention, the speed of the data may provide not only the state at a particular time, but also a rate by testing dynamically over a comparatively short period of time as the process is underway.

Cancer is characterized by abnormalities in cell structures. However, science has been developing additional characterizations of, for example, neuro-degenerative diseases, such as Alzheimer's Disease, Parkinson's disease, ALS, MS, and so forth. In those pathologies, a method 320 in accordance with the invention, may be applied and characterize any cyto-skeletal dysfunction that is characteristic of such a pathology. Thus, any cellular, cyto-skeletal dysfunction capable of causing, reflecting, or otherwise characterizing any particular pathology, may be detected by a process 320 in accordance with the invention.

As a practical matter, certain neuro-degenerative diseases are difficult to detect. For example, Alzheimer's disease is not actually detectable during the life of the patient. Typically, pathological analysis has been a result of autopsies of the brain of deceased patients. In a method 320 and with apparatus in accordance with the invention, one may detect nondestructively such pathologies.

Perhaps most importantly, one may detect pathologies nondestructively over a period of time. Thus, not only the detection of the pathology, but also detection of response of the pathology to a particular medicament, regimen, therapy, or the like may also be tested. Thus, the efficacy of therapies and drugs in containing, reversing, or slowing the progress of pathology are now enabled.

Thus, by relying on the pathologies that have already been correlated 332, the process 320 has enabled the detection of pathology and the determination of efficacy of treatments.

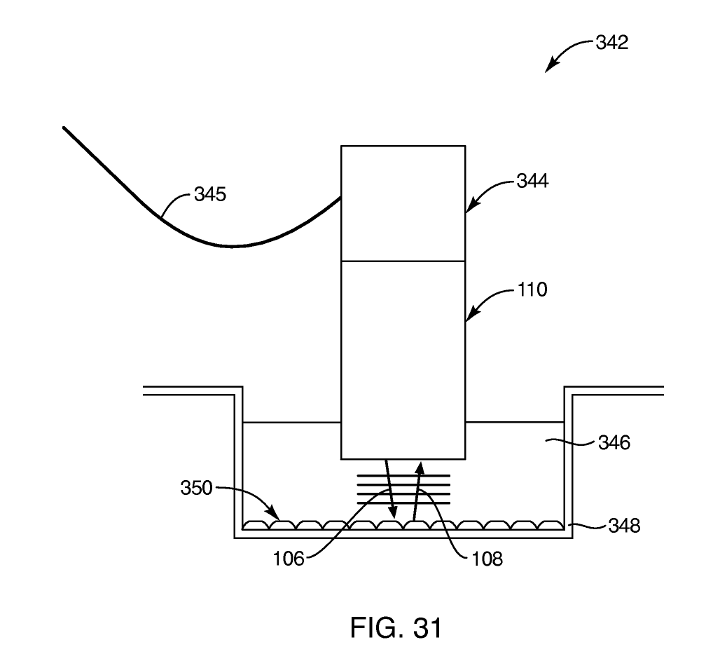

Referring to FIG. 31, an apparatus 342 or system 342 is illustrated similarly to those described hereinabove. For example, in a system 342, a connector 344 may form an interface between a cable 345 representing the exchange of power, data, and so forth with an ultrasonic transducer 110. The transducer 110 is in contact with a growth medium 346 in a tissue culture well 348. Typically, the growth medium 346 may be disposed within the well 348, and the cell growth 350 or cells 350 in a monolayer 350 may be grown thereon. Thus, a pulse 106 may be transmitted from the transducer 110 through the medium 346 to be scattered by the cells 350 or monolayer 350. The return pulse 108 may thus be detected as described in detail hereinabove.

The empirical or experimental apparatus 342 has been configured and tested. Likewise, such a system 342 has been modeled. For example, by varying size of cells and their location and distribution in a cell layer, the monolayer of cells 350 has been modeled. A random cell packing model that relies on a distribution of cells size and locations may be used to simulate a scattering of a high frequency ultrasonic pulse.

A model was created using Monte Carlo algorithm for distributing the cells by space and size. Cells and nuclei were modeled as layered spheres. A core is a nucleus, and the shell was a cytoplasm. This model provided a cell structure to simulate the ultrasonic spectra. It provides a useful model of a single layer, and may even be applied to overgrown or thicker monolayer cultures.

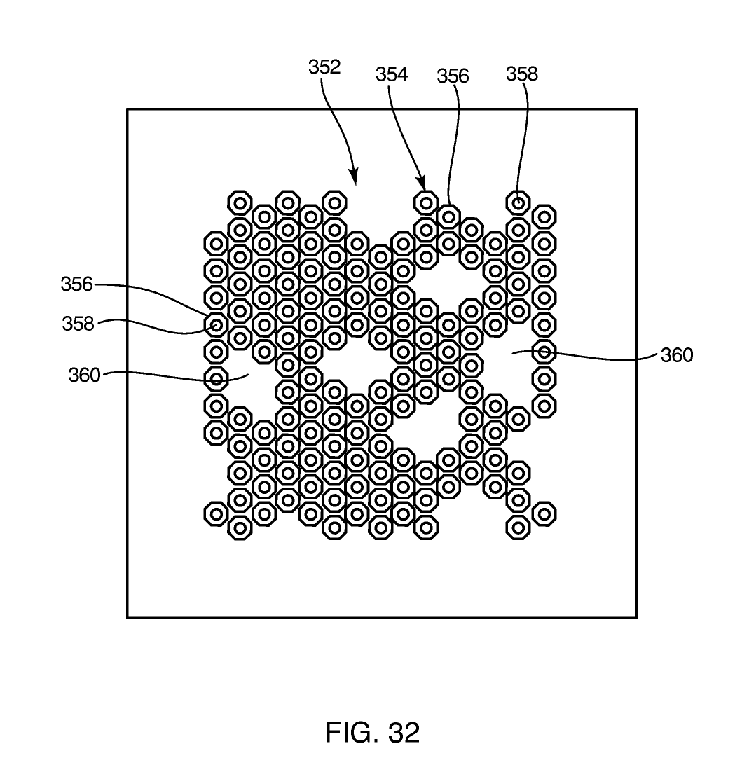

Referring to FIG. 32, a monolayer 350 cell culture has been embodied in a model 352. In that model 352 of the cells 350, an individual cell 354 is characterized by a wall 355 or cytoskeletal structure. This may be represented by the properties of the contained cytoplasm, the actual cytoskeletal structure, or both. Meanwhile, nuclei 358 are embodied in each individual cell 354.

Notably, certain gaps 360 or unoccupied spaces 360, wherein a matrix material may exist, but cells 354 are absent, has also been modeled. This model was run and provided validation that in the sizes and frequencies of interest, the modeling could accurately provide power spectra for the characteristics of cell properties of interest. Thus, changing a biomechanical property of a particular artifact within a cell, such as a nucleus, cytoplasm, cytoskeletal structure, or the like resulted in a change in the power spectra predicted by the model as a result thereof. Thus, the dependent variable of power spectrum responded affirmatively and significantly to a change of the independent variable of material property or other microstructure or property. Thus, the model was validated that it was capable of accurately representing molecular response and cellular response to ultrasonic pulses.

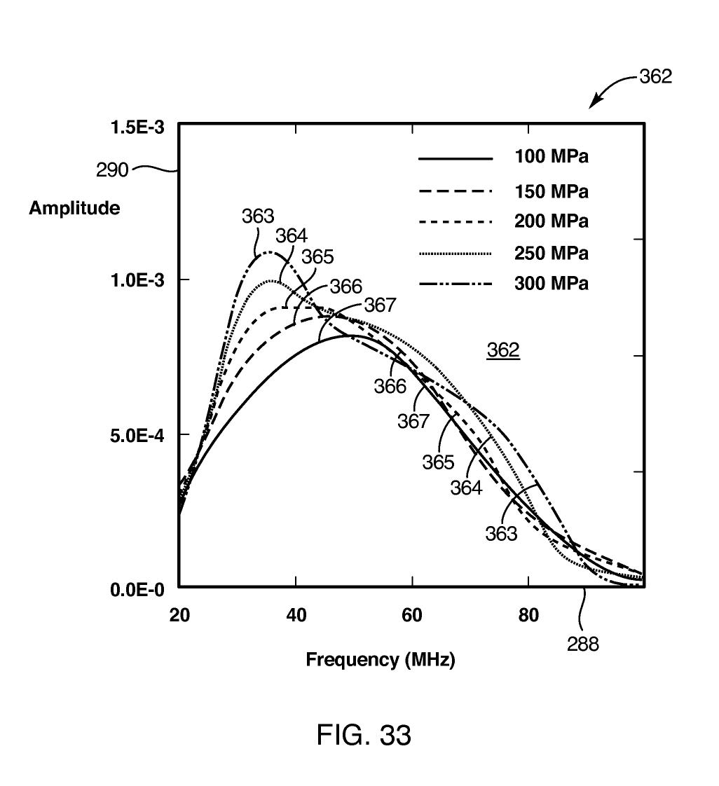

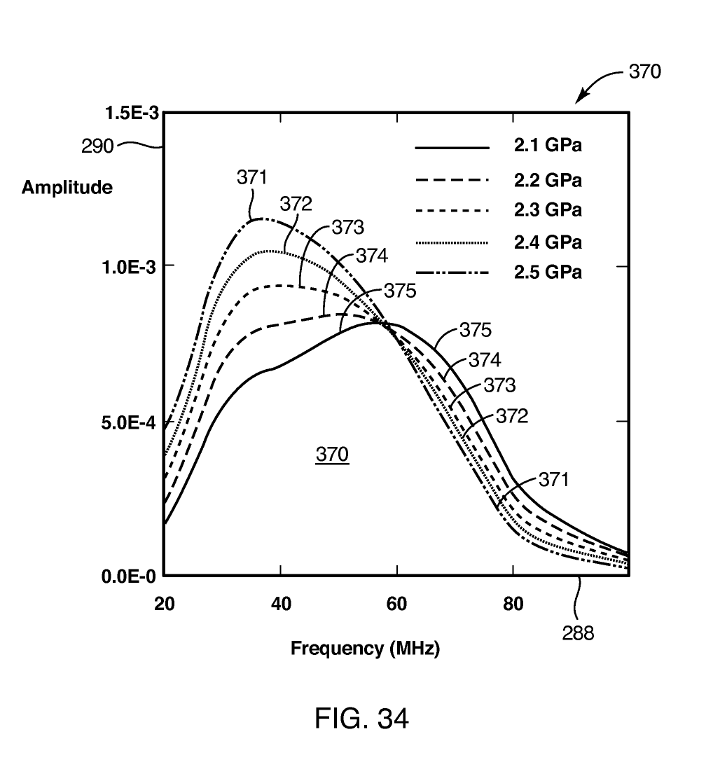

Referring to FIGS. 33 and 34, the charts 362, 370 represent validation of the model 352. For example, the chart 362 contains several curves 363, 364, 365, 366, 367. Those curves 363, 364, 365, 366, 367 represent various levels of shear modulus or values of shear modulus in the cytoplasm of a cell 354 of the model 352. One immediately notices that in the frequency domain 288 or x axis 288 the amplitude 290 provides a shape, peak height, peak location, and so forth that uniquely characterize each succeeding value of shear modulus.

By the same token, the chart 370 illustrates curves 371, 372, 373, 374, and 375. Each of these curves, represents a characteristic bulk modulus property of the cytoplasm of a cell 354 in the model 352. Again, distinctive shapes, peaks, peak locations, widths, and so forth characterize each particular bulk modulus value as measured on the y axis 290.

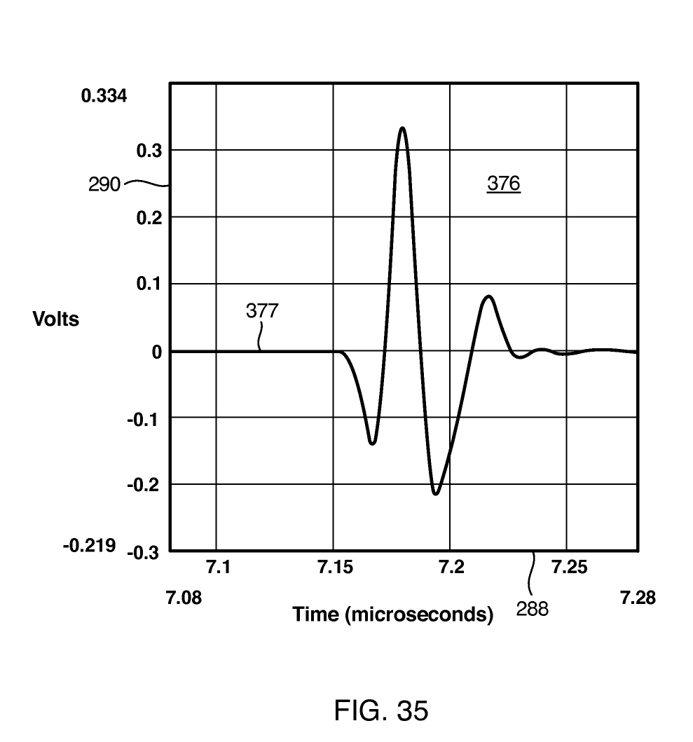

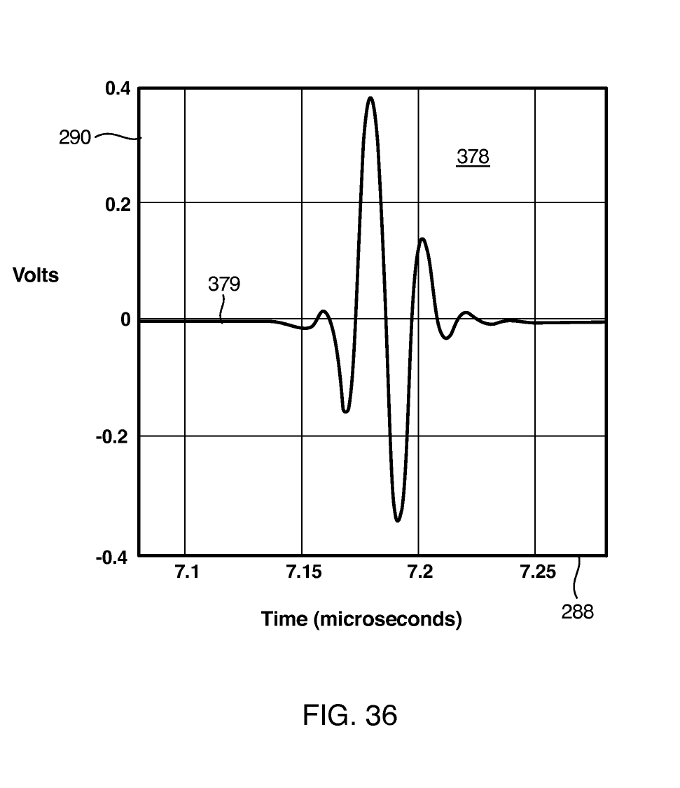

Referring to FIGS. 35 and 36, empirical data is embodied in the charts 376, 378. In these charts 376, 378, curves 377, 379 represent the response of the piezoelectric receiving transducer 114. In this embodiment, an ultrasonic pulse-echo waveform 106 was propagated through a cell culture 350 maintained in a well 348. The curve 377 represents a reflection 108 of an actual pulse 106, in a controlled configuration. That is, the curve 377 represents the reflection 108 of the applied waveform 106 from the bottom or floor of the well 348, through the culture medium, but absent any actual cell layer 350.

In contrast, the curve 379 represents the voltage on the y axis 290 across a time domain on the x axis 288 in microseconds. This is an actual response of a layer of cells 350, as well as the necessary presence of the well 348.

In comparing the curves 377, 379, one may extract or delete the superposition contribution of the curve 377 from the curve 379. One notices that at the location of approximately 7.15 micro seconds on the x axis 288, an artifact characterizing the cell layer 350 appears. Thus, one may detect a monolayer 350 of cells in an actual sample, at a resolution sufficient to apply the characterizations of FIGS. 33 and 34.

As a practical matter, the curve 379 is sufficiently clear (e.g., comparatively good signal-to-noise ratio) that the windowing step 325 of the process 320 may narrow the region of interest to that region around the 7.15 micro seconds position. Thus, there is no need to further apply the process 320 to the remainder of the trace 379 much beyond the 7.15 micro second position. Thus, examination may be more efficient, and the waveforms may be characterized in greater detail.

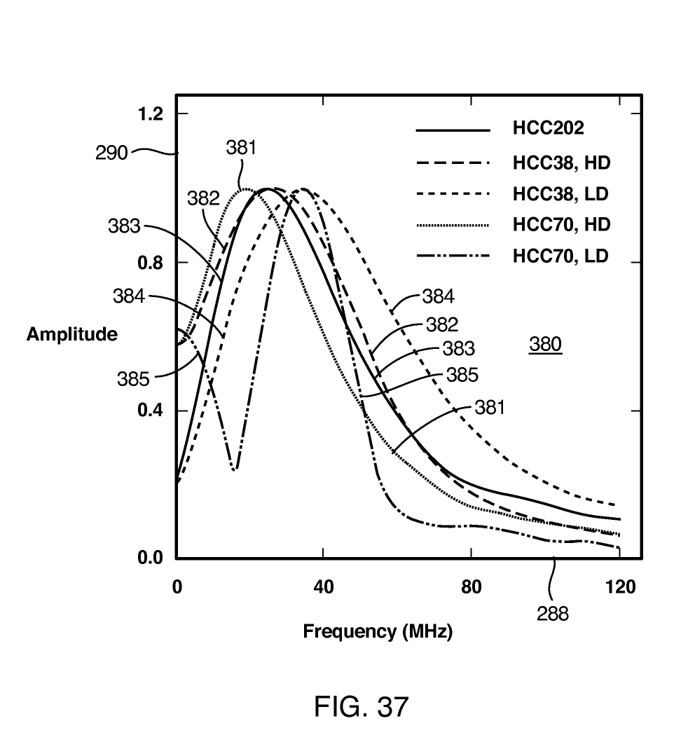

Referring to FIG. 37, a chart 380 presents the curves 381, 382, 383, 384, 385 representing various cell lines. The HCC represents the specific source (laboratory or patient resource) of the breast cancer cells. Meanwhile, the trailing digits (e.g., 202, 38, and 70) represent specific cell lines within the HCC source type.

The HCC refers to the source of a particular cell line. The trailing numbers 202, 38, 70 refer to specific cell lines originating from the same source. Meanwhile, the HD and LD designations reflect whether the cell culture 350 was developed at a low density or high density population. In the chart 380, one immediately notices that, for example, the curve 385 is much narrower than the other curves 381, 382, 383, 384. However, the peak height of all the curves 381, 382, 383, 384, 385 is very nearly the same. These are normalized peaks, but nevertheless, the fact that the normalized peaks end up at the same value renders the peak height of comparatively little value.

However, in contrast, note the specific peak location as measured along the frequency domain 288 or the x axis 288. The curve 384 and the curve 385 have approximately the same normalized peak height, and the same peak location. However, one is much wider, and the other is narrower, yet more active having more peaks and valleys. Similarly, the curves 381, 382 are similar, but shift in peak location, and in overall width. Thus, the process 320 in accordance with the invention rapidly, and distinctively identifies each of the cell lines 381, 382, 383, 384, 385 individually and distinguishably from one another.

The present invention may be embodied in other specific forms without departing from its purposes, functions, structures, or operational characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. A method for distinguishing materials, the method comprising:
providing a device comprising an ultrasonic transducer corresponding to a range of frequencies;
selecting a specimen;
selecting a waveform;
applying to the specimen, by the at least one ultrasonic transducer, an applied pulse characterized by an ultrasonic frequency and corresponding to the waveform;
transmitting the applied pulse through the specimen;
detecting a received pulse corresponding to distortions to the applied pulse by the specimen after passing into the specimen;
analyzing the received pulse by
computing a simulation of the received pulse by a numerical method based on the microstructure of the specimen,
generating a first spectrum, by transforming the simulation to a frequency domain,
generating a second spectrum by transforming the received pulse from a time domain to a frequency domain, and
comparing, by an artificial intelligence engine, the first spectrum to the second spectrum; and
classifying the specimen, based on the comparison.

2. The method of claim 1, wherein the artificial intelligence engine is selected from:
a principal component analysis;
a signal interpretation engine;
a pattern recognition program;
a correlation, for one or more features of the first and second spectra, of at least one of amplitude, other magnitude, average position in the domain, peak position in the domain, extent along a domain axis, slope, average value, other weighted value, and integral of the one or more features.

3. The method of claim 2, wherein the range is from about 10 to about 100 megahertz.

4. The method of claim 3, wherein the applied pulse passes through the specimen at least once.

5. The method of claim 3, wherein the applied pulse:
passes through the specimen;
reflects; and
returns back through the specimen to a position proximate the transmitter to be detected as the received pulse.

6. The method of claim 3, wherein:
the ultrasonic transducer comprises at least one ultrasonic transducer; and
the applied pulse is generated by a transmitter, of the at least one transducer, and the pulse is detected by a receiver, of the at least one transducer.

7. The method of claim 6, wherein the distance through the specimen is sized to be less than 5 millimeters along the direction of travel of the applied pulse.

8. The method of claim 7, wherein:
the distance is less than about two millimeters.

9. The method of claim 6, wherein:
the at least one ultrasonic transducer comprises a transmitter and a receiver;
the pulse is generated by the transmitter;
the pulse is reflected from an anvil; and
the pulse is detected by the receiver.

10. The method of claim 1, wherein:
the at least one ultrasonic transducer comprises a first, transmitter, ultrasonic transducer and a second, receiver, ultrasonic transducer;
the pulse is transmitted through the specimen at least once;
the specimen comprises cells corresponding to an animal;
the at least one ultrasonic transducer is embedded in an instrument selected from a scalpel, needle, probe, and container.

11. A method for determining molecular subtypes, the method comprising:
providing a specimen;
providing at least one ultrasonic transducer;
selecting a waveform;
applying to the specimen, by the at least one ultrasonic transducer, an applied pulse characterized by an ultrasonic frequency and corresponding to the waveform;
transmitting the applied pulse through the specimen;
detecting, by the at least one ultrasonic transducer, a received pulse corresponding to the applied pulse and the specimen;
analyzing the received pulse by
computing a simulation of the received pulse by a numerical method based on the microstructure of the specimen,
generating a first spectrum, by transforming the simulation to a frequency domain,
generating a second spectrum by transforming the received pulse from a time domain to a frequency domain, and
comparing, by an artificial intelligence engine, the first spectrum to the second spectrum; and
classifying the specimen, based on the comparison.

12. The method of claim 11, wherein the artificial intelligence engine is selected from:
a principal component analysis;
a signal interpretation engine;
a pattern recognition program;
a correlation, for one or more features of the first and second spectra, of at least one of amplitude, other magnitude, average position in the domain, peak position in the domain, extent along a domain axis, slope, average value, other weighted value, and integral of the one or more features.

13. The method of claim 12, wherein the ultrasonic frequency is from about 10 to about 100 megahertz.

14. The method of claim 13, wherein the applied pulse passes through the specimen once before being received as the received pulse.

15. The method of claim 13, wherein the applied pulse:
passes through the specimen;
reflects; and
returns back through the specimen to a position proximate the transmitter to be detected as the received pulse.

16. The method of claim 13, wherein the pulse is generated by a transmitter, of the at least one transducer, and the pulse is detected by a receiver, of the at least one transducer.

17. The method of claim 16, wherein the specimen is sized to be less than about 15 millimeters through along the direction of travel of the applied pulse.

18. The method of claim 16, wherein:
the at least one ultrasonic transducer comprises a transmitter and a receiver;
the pulse is generated by the transmitter;
the pulse is reflected from an anvil; and
the pulse is detected by the receiver.

19. The method of claim 11, wherein:
the at least one ultrasonic transducer comprises a first, transmitter, ultrasonic transducer and a second, receiver, ultrasonic transducer;
the pulse is transmitted through the specimen at least once;
the specimen comprises cells corresponding to an animal;
the at least one ultrasonic transducer is embedded in an instrument selected from a scalpel, needle, probe, and container.

20. The method of claim 11, 21. The method of claim 11, wherein:
the specimen comprises cells or other particulate material in fluid suspension held in a container;
the method further comprises acoustically levitating a layer of at least one of cells and particulate material by a standing wave in response to an ultrasonic wave propagated at a frequency of from about 20 to about 500 kilohertz;
the method further comprises the at least one ultrasonic transducer delivering an applied pulse at a frequency in a range of from about 10 to about 100 megahertz;
the method further comprises reflecting, by the applied pulse, from the layer; and
detecting, as the received pulse, by the at least one transducer the applied pulse as altered by the reflecting.
